(19) United States
(12) Patent Application Publication
(10) Pub. No.: US 2008/0045499 A1
(43) Pub. Date: Feb. 21, 2008

(54) Preferential Inhibition of Presenilin-1

(75) Inventors: Byron B. Zhao, Sugar Land, TX (US); Mei Yu, San Francisco, CA (US); Guriqbal S. Basi, Palo Alto, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Elan Pharmaceuticals, Inc.

(21) Appl. No.: 11/671,926

(22) Filed: Feb. 6, 2007

Related U.S. Application Data
(60) Provisional application No. 60/745,344, filed on Apr. 21, 2006.
(60) Provisional application No. 60/771,117, filed on Feb. 6, 2006.

(51) Int. Cl. A61K 31/18; A61K 31/55; A61P 25/28; C07C 311/16; C07D 223/10; C07D 401/12; C07K 14/00; C07K 16/00; C12Q 1/02

(52) U.S. Cl. 514/212.03; 435/29; 514/212.04; 514/604; 514/789; 530/324; 530/387.9; 530/389.1; 540/522; 540/524; 540/527; 564/92

(57) ABSTRACT

The invention provides methods for determining whether an agent preferentially inhibits Presenilin-1-comprised γ-secretase relative to Presenilin-2-comprised γ-secretase. The invention also provides agents that preferentially inhibit Presenilin-1-comprised γ-secretase relative to Presenilin-2-comprised γ-secretase, pharmaceutical compositions comprising such compounds, and methods of treating Alzheimer's disease using such compounds. The invention also discloses that the N-terminal domain of presenilin-1 and -2 determines the difference in the production of Aβ by PS1-comprised and PS2-comprised gamma secretases. This finding identified the structural determinant for the observed difference in the production of Aβ by PS1-comprised and PS2-comprised gamma secretases. Such structural determinant was not identified before. This invention also provides a method for determining whether an agent specifically binds the N terminus of PS1. The invention further provides for methods of treatment of Alzheimer's Disease by administration of an effective dose of an agent which specifically binds PS1, thereby inhibiting PS1 activity.

This application claims the benefit of U.S. Provisional Patent Application No. 60/771,117 filed Feb. 6, 2006 and U.S. Patent Application No. 60/745,344 filed Apr. 21, 2006, the disclosures of each of which are incorporated herein by reference in their entirety.

### FIELD OF THE INVENTION

The invention relates to methods for identifying compounds that preferentially inhibit Presenilin-1-comprised γ-secretase relative to Presenilin-2-comprised γ-secretase. The invention also relates to agents that preferentially inhibit Presenilin-1-comprised γ-secretase relative to Presenilin-2-comprised γ-secretase, pharmaceutical compositions comprising such compounds, and methods of treating Alzheimer's disease using such compounds and pharmaceutical compositions.

The invention further relates to agents that interact specifically with the N-terminal portion of PS1 thereby preferentially inhibiting PS1 relative to PS2. The invention also relates to pharmaceutical compositions comprising such agents, methods of preferentially inhibiting PS1 relative to PS2 in a cell, and methods of treating Alzheimer's disease using such agents and pharmaceutical compositions.

The invention further relates to identification of structural determinants for PS1 selective inhibition by some compounds that specifically inhibit PS1 comprised γ-secretase activity relative to Presenilin-2-comprised γ-secretase.

### BACKGROUND OF THE INVENTION

Alzheimer's disease (AD) is one of the most common forms of dementia, and is one of the leading causes of death in the United States. Nearly 30% of all 85-year-olds have AD (Brunkan A. L. & Goate A. M., J. Neurochem. (2005) 93:769-792) AD is characterized by neuronal cell loss and the accumulation of neurofibrillary tangles and senile plaques in the brain.

The primary cause of the senile plaques is the amyloid-β peptide (Aβ) which is produced by proteolytic processing of amyloid precursor protein (APP). APP is a ubiquitously expressed integral membrane protein which is proteolytically processed by secretases in various pathways. Cleavage of APP at the a site is benign. However, cleavage at the β and γ sites results in the formation of an Aβ peptide, which may be 40, 42 or 43 residues long.

Presenilins (PS) have been shown to form the catalytic subunit of the γ-secretase complex that produces the Aβ peptide. Most mutations in APP and PS increase the ratio of a 42-residue form of Aβ (Aβ42) versus 40-residue Aβ (Aβ40), thus defining a common AD phenotype caused by APP, PS1 and PS2 mutations (Scheuner D., et al., Nat. Med. 2:864-870). Aβ peptides ending at residue 42 or 43 (long tailed Aβ) are thought to be more fibrillogenic and more neurotoxic than Aβ ending at residue 40, which is the predominant isoform produced during normal metabolism of βAPP (St. George-Hyslop, P. H., & Petit, A., C. R. Biologies (2004) 328:119-130). The Aβ42 peptide is thought to initiate the amyloid cascade, a pathological series of neurotoxic events, which eventually leads to neurodegeneration in Alzheimer's Disease (Selkoe, D. J., J Clin Invest (2002) 110:1375-1381). Aβ promotes oxidative stress either directly or indirectly (Kanski J, et al., Neurotoxicity Research (2002) 4:219-223.

Presenilins are known to be involved in the regulation of β-catenin stability, trafficking of membrane proteins, and γ-secretase cleavage of APP and other substrates. All PS1 mutations associated with AD increase γ-secretase cleavage of βAPP and preferentially increase the production of long-tailed Aβ peptides ending at residue 42. Some believe, however, that PS2 mutations may also cause neurodegeneration by modulating cellular sensitivity to apoptosis induced by a variety of factors, including Aβ peptide. (Martins R. N., et al., (1995) High levels of amyloid beta-protein from S182 (Glu246) familial Alzheimer's cells, NeuroReport 7, 217-220; Duff K., et al., (1996) Increased amyloid beta protein 42(43) in brains of mice expressing mutant presenilin 1. Nature 383:710-713; Citron M., et al., (1997) Mutant presenilins of Alzheimer's Diease incrase production of 42 residue amyloid beta protein in both transfected cells and transgenic mice. Nat Med. 3:67-72; Rogaev E. I., et al., (1995) Familial Alzheimer's disease in kindreds with missense mutations in a novel gene on chromosome 1 related to the Alzheimer's Disease type 3 gene. Nature 376:775-778.) γ-secretase appears to be an aspartyl protease that cleaves both APP and Notch.

Most cells express both PS1-comprised γ-secretase and PS2-comprised γ-secretase, with PS1-comprised γ-secretase being primarily responsible for Aβ production and probably also Notch signaling. (Shen et al (1997) Skeletal and CNS defects in Presenilin-1-deficient mice. Cell 89:629-39; Wong et al (1997). Presenilin 1 is required for Notch1 and DII1 expression in the paraxial mesoderm. Nature 387:288-92; De Strooper et al (1998) Deficiency of presenilin-1 inhibits the normal cleavage of amyloid precursor protein. Nature 391:387-90). Notch proteins are large molecular weight cell-surface membrane receptors that mediate complex cell fate decisions during development. (Chen Q., Schubert D., (2002) Presenilin-interacting proteins. Expert Rev Mol Med. 2002:1-18.) It is also thought that γ-secretase cleaves epithelial cadherin, a type 1 transmembrane protein that mediates Ca²⁺-dependent cell-cell adhesion and recognition, ErbB-4, an epidermal growth factor that controls cell proliferation and differentiation, and CD44, another receptor that mediates cell adhesion. (Kimberly W. T., Wolfe M. S., (2003) Identity and Function of γ-secretase. J. Neuroscience Res. 74:353-260) Thus, a major challenge in developing therapeutics for treating AD has been to identify inhibitors of γ-secretase that reduce the production of amyloid peptides from APP without significantly affecting the cleavage of other γ-secretase substrates such as Notch.

Recent studies of PS1 and PS2 activity in cultured cell lines, however, indicate that even a low-level of γ-secretase activity may be sufficient to support proper functioning of γ-secretase substrates other than APP, such as Notch signaling. These studies suggest that selective inhibition of PS1-comprised γ-secretase would lead to a significant decrease in Aβ production, and that the residual γ-secretase activity of PS2-comprised γ-secretase would be sufficient to support the cleavage of other essential γ-secretase substrates such as Notch. In fact, experiments using conditional knockout mice, in which the expression of the PS1 gene in the brain has been ablated, show that such mice exhibit remarkably normal properties at anatomical, physiological, and behavioral levels. These experiments suggest that selective inhibition of PS1-comprised γ-secretase in adulthood may cause few side effects.

There is a need in the art for methods and agents that can reduce Aβ production without significantly affecting other γ-secretase substrates and pathways. One way to address this need is to identify inhibitors of γ-secretase that preferentially inhibit PS1 relative to PS2 by binding specifically to PS1. In particular there is a need in the art to identify the active region of PS1 in order to identify and/or design agents that target specifically an active region of PS1, structurally distinct from PS2. Inhibitors targeting such region may specifically inhibit PS1-comprised γ-secretase activity, but spare PS2-comprised γ-secretase activity. Therefore, the identification of a PS1 active region and inhibitors thereof would provide therapeutic candidates compounds for use in treating AD that have decreased or minimal side effect profiles.

Therefore, one possible way to reduce Aβ production without significantly affecting other γ-secretase substrates is to identify inhibitors of γ-secretase that preferentially inhibit Presenilin-1-comprised γ-secretase relative to Presenilin-2-comprised γ-secretase. The identification of such inhibitors would provide additional therapeutic candidates having acceptable side effect profiles for use in treating AD.

### SUMMARY OF THE INVENTION

The present invention provides a method for identifying a compound that preferentially inhibits Presenilin-1-comprised γ-secretase relative to Presenilin-2-comprised γ-secretase. The method comprises separately incubating a first cell type that expresses Presenilin-1 but does not express Presenilin-2 and a second cell type that expresses Presenilin-2 but does not express Presenilin-1 with the compound; determining the amount of Aβ1-x, which includes Aβ40/42, in each cell type; calculating the EC₅₀ value for Aβ1-x in each cell type; and determining that the compound preferentially inhibits Presenilin-1-comprised γ-secretase relative to Presenilin-2-comprised γ-secretase if the EC₅₀ value calculated for the first cell type is smaller than the EC₅₀ value calculated for the second cell type.

The present invention also provides compounds that preferentially inhibit Presenilin-1-comprised γ-secretase relative to Presenilin-2-comprised γ-secretase, pharmaceutical compositions for treating Alzheimer's disease comprising a non-toxic therapeutically effective amount of a compound that preferentially inhibits Presenilin-1-comprised γ-secretase relative to Presenilin-2-comprised γ-secretase and a pharmaceutically acceptable carrier, and methods of treating Alzheimer's disease comprising administering to a patient in need of treatment a pharmaceutical composition comprising a non-toxic therapeutically effective amount of a compound that preferentially inhibits Presenilin-1-comprised γ-secretase relative to Presenilin-2-comprised γ-secretase and a pharmaceutically acceptable carrier.

In one aspect, the invention provides presenilin 1-comprised gamma secretase (PS1) specific binding agents that can modulate PS1 biological activity.

In an aspect, the invention relates to compositions comprising PS1 specific binding agents and pharmaceutically acceptable salts thereof.

In another aspect, the invention provides methods for specifically inhibiting PS1, comprising contacting PS1 with a PS1 specific binding agent that binds to the N-terminal third of PS1 (amino acid residues 1-127; SEQ ID NO: 8) in an amount effective for specific inhibition.

In another aspect, the invention provides structural determinants for PS1 selective inhibition by small molecule inhibitors of PS1 gamma secretas. More specifically, the invention provides structural determinants for PS1 responsible for differential inhibition of PS1 gamma secretase activity by small molecule inhibitors. The invention further demonstrates that selective inhibitors of PS1 interact with the middle 1/3 portion of PS1 (residues 128-298) (SEQ ID NO: 9), more specifically residues L172, T281 and T282.

In another aspect, the invention provides method of treating or preventing Alzheimer's disease (AD) in a subject comprising administering to the subject an amount effective to treat or prevent AD of a PS1 specific binding agent, or pharmaceutically acceptable salts thereof.

In a further aspect the invention relates to methods for inhibiting the production of A-beta (Aβ) in a cell comprising contacting a cell with a PS1 specific binding agent in an amount effective to inhibit PS1 gamma secretase activity but not inhibit PS2 gamma secretase activity.

In yet another aspect, the invention provides for an isolated polypeptide comprising the terminal third of PS1, the N terminal 127 amino acids (SEQ ID NO: 8).

Specific embodiments of the present invention will become evident from the following detailed description of the invention and the appended claims.

### BRIEF DESCRIPTION OF THE DRAWINGS

FIG. 1A-1C represents the Presenilin-1 (PS1) amino acid sequence (SEQ ID NO:2) and a nucleic acid sequence (SEQ ID NO:1) that codes for the PS1 amino acid sequence.

FIG. 2A-2C represents the Presenilin-2 (PS2) amino acid sequence (SEQ ID NO:4) and a nucleic acid sequence (SEQ ID NO:3) that codes for the PS2 amino acid sequence.

FIG. 3 represents the Aβ43 (Aβ43) amino acid sequence (SEQ ID NO: 5).

FIG. 4 represents the amino acid sequence for the Swedish Mutation Amyloid Precursor Protein (APPswe) (SEQ ID NO: 6).

FIG. 5 provides the sequence origin of PS1/PS2 chimeras, and represents the determination of relative protein expression levels for different chimeras.

FIG. 6 shows the determination of relative activity of various presenilin constructs illustrated in FIG. 5.

FIG. 7 represents the chimeric PS1/PS2 molecules used to determine which segment(s) of PS1 and PS2 are most responsible for Aβ production. This demonstrates that, PS12A, PS12B, and PS12C had similar activity as PS1, while PS21A, and PS21C had similar activity as PS2, and PS12D and PS21D are intermediate between PS1 and PS2, thus leading to the conclusion that the N-terminal third of PS1 conferred a high relative activity, with the first half (amino acid residues 1-70 in PS1) to be slightly more important than the second half (amino acid residues 71-127 in PS1) of this region. Although data on PS21F may suggest that the N-terminal sixth accounts for the entire contribution to activity by the N-terminal third, data from PS12D and PS21D chimeras contradict this observation. So overall, it is the N-terminal third (amino acid residues 1-127 in PS1) that appear to possess an almost full ability to stimulate γ-secretase activity.

FIG. 8 represents the Presenilin-1 (PS1) amino acid sequence (SEQ ID NO: 9) that codes for the middle third portion of PS1.

FIG. 9 is Dose Response curves and EC50 values from experiments of different compounds for inhibition of PS1-γ-secretase

FIG. 10 is a map of Chimeric PS1/PS2 molecules.

FIG. 11 is a table showing the mean values from 2 independent experiments on PS1/PS2 selectivity of various inhibitors.

### DETAILED DESCRIPTION OF THE INVENTION

The section headings are used herein for organizational purposes only, and are not to be construed as in any way limiting the subject matter described. All references cited herein are incorporated by reference in their entirety.

Standard techniques may be used for recombinant DNA molecule, protein, and antibody production, as well as for tissue culture and cell transformation. See, e.g., Sambrook, et al. (below) or Current Protocols in Molecular Biology (Ausubel et al., eds., Green Publishers Inc. and Wiley and Sons 1994). Enzymatic reactions and purification techniques are typically performed according to the manufacturer's specifications or as commonly accomplished in the art using conventional procedures such as those set forth in Sambrook et al. (Molecular Cloning: A Laboratory Manual. Cold Spring Harbor Laboratory Press, Cold Spring Harbor, N.Y. (1989)), or as described herein. Unless specific definitions are provided, the nomenclature utilized in connection with, and the laboratory procedures and techniques of analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

In one aspect the invention provides a method for identifying a compound that preferentially inhibits Presenilin-1-comprised γ-secretase relative to Presenilin-2-comprised γ-secretase. The method comprises (a) separately incubating with a compound a first cell type and a second cell type, wherein the first cell type expresses Presenilin-1 but does not express Presenilin-2, and the second cell type expresses Presenilin-2 but does not express Presenilin-1; (b) determining the amount of Aβ1-x, which includes Aβ40 and Aβ42, in each cell type (c) calculating the EC₅₀ value for Aβ1-x in each cell type; and (d) determining that the compound preferentially inhibits Presenilin-1-comprised γ-secretase relative to Presenilin-2-comprised γ-secretase if the EC₅₀ value calculated for the first cell type is smaller than the EC₅₀ value calculated for the second cell type.

In certain embodiments of this aspect, the compound “preferentially” inhibits Presenilin-1-comprised γ-secretase relative to Presenilin-2-comprised γ-secretase when the ratio of the EC₅₀ value for the cell comprising Presenilin-2-comprised γ-secretase to the EC₅₀ value for the cell comprising Presenilin-1-comprised γ-secretase is greater than 1. In a preferred embodiment, the ratio of the EC₅₀ value is about 3-5, more preferably about 5-10, even more preferably about 10-15, yet more preferably about 15-20, and most preferably greater than about 20.

A. Definitions

As used herein, the term “specific binding agent” refers to a molecule or molecules that have specificity for recognizing and binding PS1 as described herein. Suitable specific binding agents include, but are not limited to, antibodies and derivatives thereof, polypeptides (such as antibodies), compounds (such as chemical compounds), and small molecules. Suitable specific binding agents may be prepared using methods known in the art, and as described herein. A PS1 specific binding agent of the invention is capable of binding a certain portion of PS1, and preferably modulating the activity or function of PS1. An exemplary PS1 specific binding agent of the invention is capable of preferentially binding to a certain portion of PS1 relative to PS2, and preferably modulating the activity or function of PS1 and not modulating the activity or function of PS2.

As used herein, the term “small molecule” refers to a molecule that has a molecular weight of less then about 1500 g/Mol. A small molecule can be, for example, small organic molecules, peptides or peptide-like molecules.

The term “antibody” as used herein refers to a monomeric or multimeric protein comprising one or more polypeptide chains that can bind specifically to an antigen and may be able to inhibit or modulate the biological activity of the antigen. The terms as used herein thus include an intact immunoglobulin of any isotype, or a fragment thereof that can compete with the intact antibody for specific binding to the target antigen, and includes, for example, chimeric, humanized, fully human, and bispecific antibodies. An intact antibody generally will comprise at least two full-length heavy chains and two full-length light chains, but in some instances may include fewer chains such as antibodies naturally occurring in camelids that may comprise only heavy chains. Antibodies may be derived solely from a single source, or may be “chimeric,” that is, different portions of the antibody may be derived from two different antibodies. For example, the CDR regions may be derived from a rat or murine source, while the framework region of the V region are derived from a different animal source, such as a human. Antibodies or binding fragments as described herein may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Thus, the term includes a polypeptide that comprises all or part of a light and/or heavy chain variable region that can bind specifically to an antigen (e.g., glucagon). The term antibody thus includes immunologically functional fragments and include, for instance, F(ab), F(ab′), F(ab′)₂, Fv, and single chain Fv fragments.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. Preferably, the antigen used herein comprises the N terminal 127 amino acids of PS1, or any suitable portion thereof capable of producing antibodies in an animal. In certain embodiments, the antigen comprises at least five contiguous amino acids contained at least in part in the amino terminus (amino acids 1-127) of PS1, such as amino acids 1-5, 2-6, 3-7, 4-8, 5-9, 6-10, 7-11, 8-12, 9-13, 10-14, 11-15, 12-16, 13-17, 14-18, 15-19, 16-20, 17-21, 18-22, 19-23, 20-24, 21-25, 22-26, 23-27, 24-28, 25-29, 26-30, 27-31, 28-32, 29-33, 30-34, 31-35, 32-36, 33-37, 34-38, 35-39, 36-40, 37-41, 38-42, 39-43, 40-44, 41-45, 42-46, 43-47, 44-48, 45-49, 46-50, 47-51, 48-52, 49-53, 50-54, 51-55, 52-56, 53-57, 54-58, 55-59, 56-60, 57-61, 58-62, 59-63. 60-64, 61-65, 62-66, 63-67, 64-68, 65-69, 66-70, 67-71, 68-72, 69-73, 70-74, 71-75, 72-76, 73-77, 74-78, 75-79, 76-80, 77-81, 78-82, 79-83, 80-84, 81-85, 82-86, 83-87, 84-88, 85-89, 86-90, 87-91, 88-92, 89-93, 90-94, 91-95, 92-96, 93-97, 94-98, 95-99, 96-100, 97-101, 98-102, 99-103, 100-104, 101-105. 101-105, 102-106, 103-107, 104-108, 105-109, 106-110, 107-111, 108-112, 109-113, 110-114, 111-115, 112-116, 113-117, 114-118, 115-119, 116-120, 117-121, 118-122, 119-123, 120-124, 121-125, 122-126, 123-127, 124-128, 125-129, 126-130, or 127-131.

“Specific binding” as used herein relates to the interaction between two different molecules, having an area on the surface or in a cavity that specifically binds to and is thereby defined as complementary with a particular spatial and physical organization of the other molecule. Types of molecules that exhibit specific binding can be referred to as ligand and receptor (antiligand). Such molecules can be members of an immunological pair such as antigen-antibody, although specific binding can occur between other molecules. As such, “specific binding” can be defined by the binding constant of two (or more) molecules.

B. Specific Binding Agents

In certain embodiments, the invention provides presenilin 1-comprised gamma secretase (PS1) specific binding agents that can modulate PS1 biological activity. In particular embodiments the specific binding agents bind to the N-terminal portion of PS1. In an aspect of this embodiment the specific binding is to the N-terminal portion of PS1, and not to the N-terminal portion of presenilin 2-comprised gamma secretase (PS2).

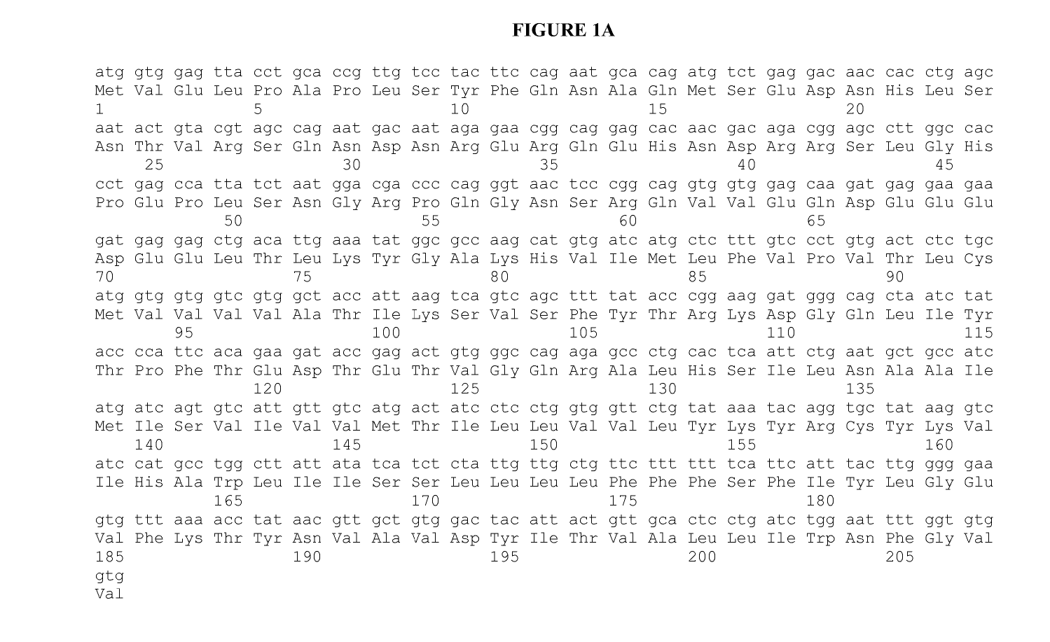

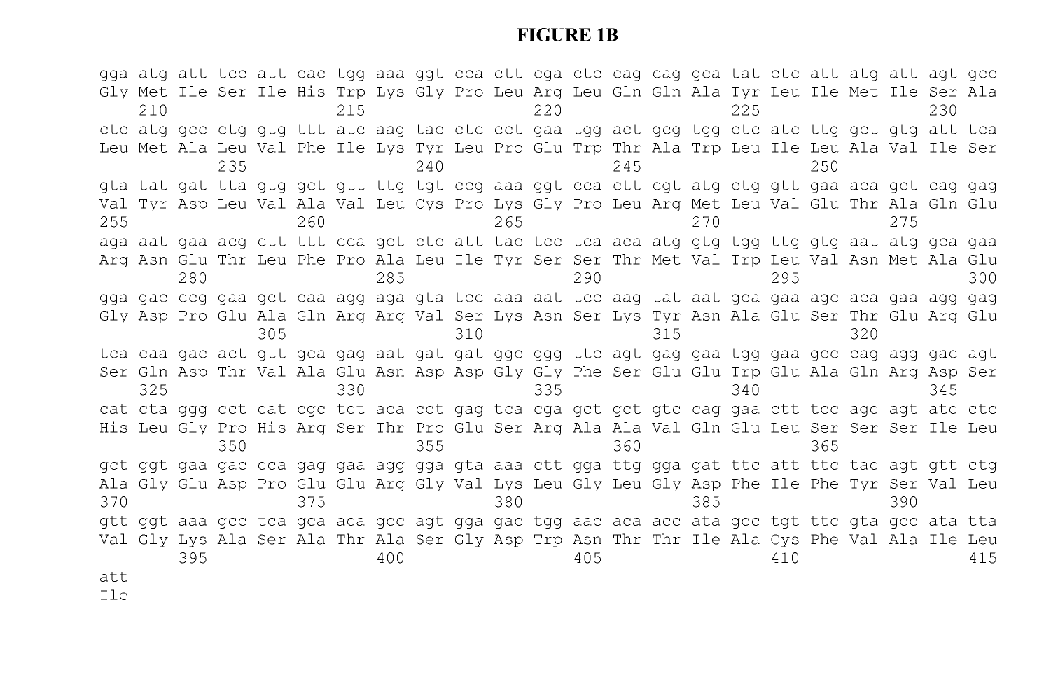

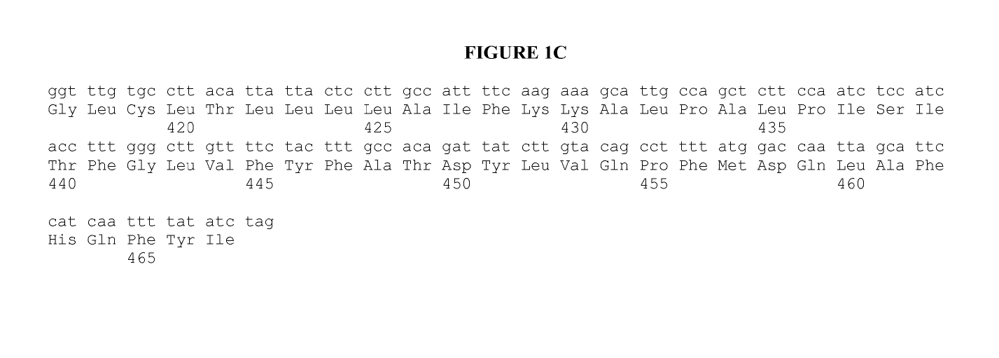

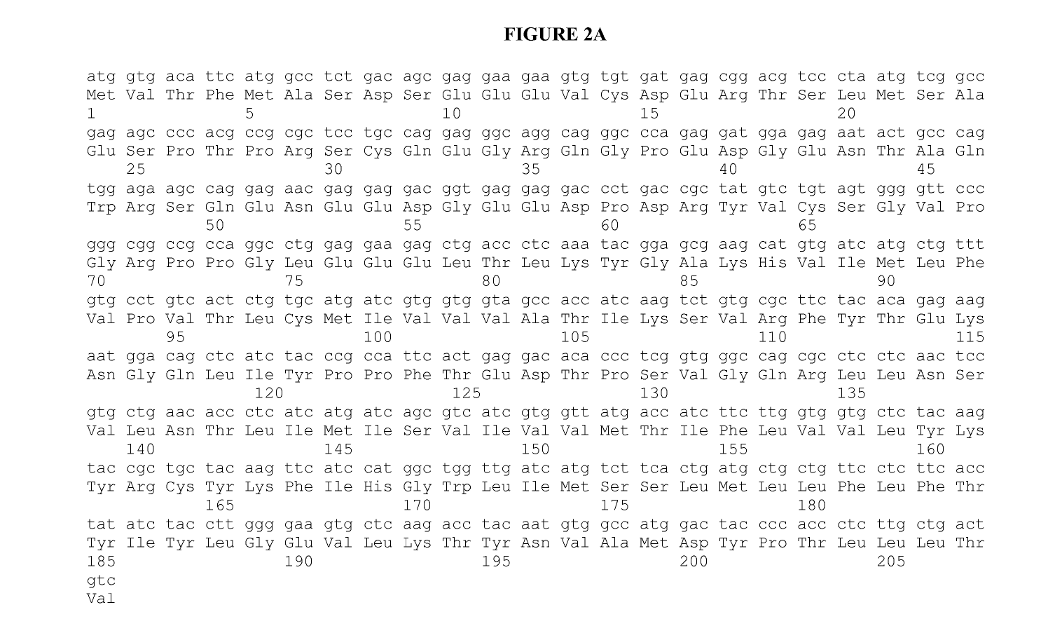

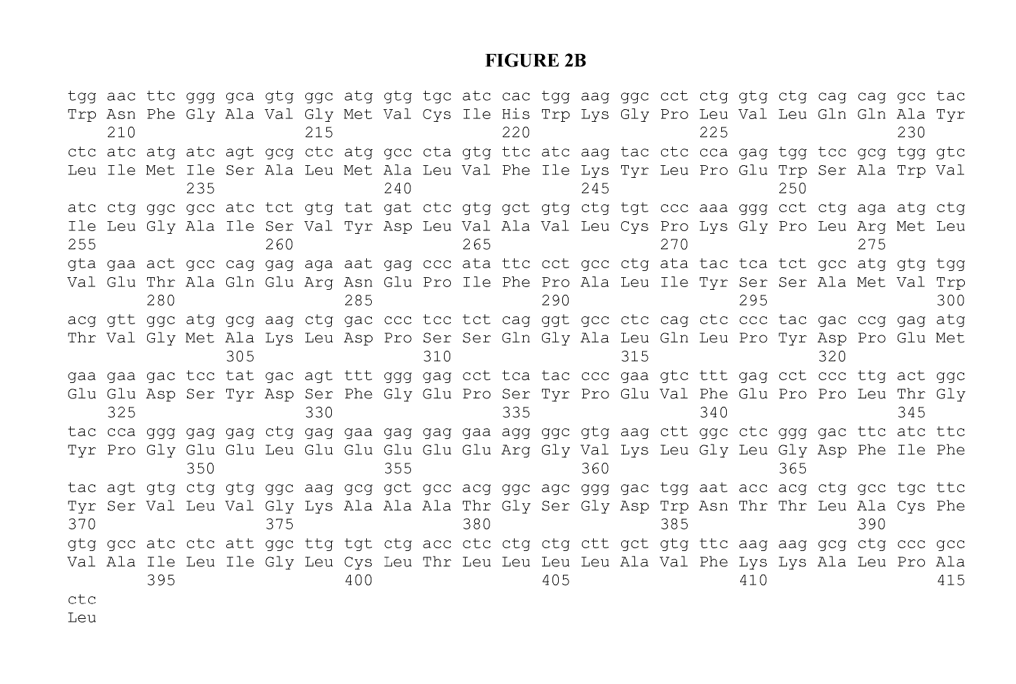

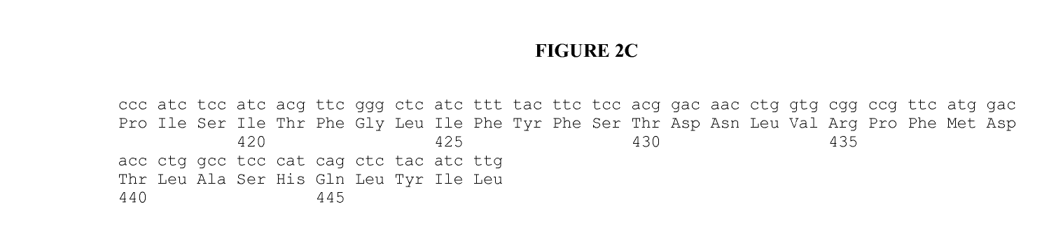

In another embodiment, the specific binding agent comprises at least one peptide having specific binding activity for PS1 or a fragment thereof. In a preferred embodiment the specific binding agent comprises at least one peptide having specific binding activity to SEQ ID NO: 2 or a fragment thereof. In one preferred embodiment, the specific binding agent is an antibody. A preferred antibody of this embodiment will recognize the N-terminal portion of PS1. More preferably, the antibody will recognize and bind to the amino acid sequence of SEQ ID NO: 8, i.e. the first 127 amino acids of PS1 (see FIG. 1.) The preferred antibody will recognize an epitope of at least five contiguous amino acids contained at least in part in the amino terminus (amino acids 1-127) of PS1 (SEQ ID NO: 8). In preferred embodiments of the present invention, the antibody recognizes at least amino acids 1-5, 2-6, 3-7, 4-8, 5-9, 6-10, 7-11, 8-12, 9-13, 10-14, 11-15, 12-16, 13-17, 14-18, 15-19, 16-20, 17-21, 18-22, 19-23, 20-24, 21-25, 22-26, 23-27, 24-28, 25-29, 26-30, 27-31, 28-32, 29-33, 30-34, 31-35, 32-36, 33-37, 34-38, 35-39, 36-40, 37-41, 38-42, 39-43, 40-44, 41-45, 42-46, 43-47, 44-48, 45-49, 46-50, 47-51, 48-52, 49-53, 50-54, 51-55, 52-56, 53-57, 54-58, 55-59, 56-60, 57-61, 58-62, 59-63. 60-64, 61-65, 62-66, 63-67, 64-68, 65-69, 66-70, 67-71, 68-72, 69-73, 70-74, 71-75, 72-76, 73-77, 74-78, 75-79, 76-80, 77-81, 78-82, 79-83, 80-84, 81-85, 82-86, 83-87, 84-88, 85-89, 86-90, 87-91, 88-92, 89-93, 90-94, 91-95, 92-96, 93-97, 94-98, 95-99, 96-100, 97-101, 98-102, 99-103, 100-104, 101-105. 101-105, 102-106, 103-107, 104-108, 105-109, 106-110, 107-111, 108-112, 109-113, 110-114, 111-115, 112-116, 113-117, 114-118, 115-119, 116-120, 117-121, 118-122, 119-123, 120-124, 121-125, 122-126, 123-127, 124-128, 125-129, 126-130, or 127-131.

In another embodiment, the specific binding agent comprises a small molecule having specific binding activity for PS1. In a preferred embodiment the small molecule specifically binds to the N-terminal portion of PS1 relative to the N-terminal portion of PS2.

In various embodiments, the invention provides methods for identification of a specific binding agent that preferentially inhibits PS1-comprised γ-secretase relative to PS2-comprised γ-secretase and/or identification of a known specific binding agent for a novel use (i.e., preferential inhibition of PS1-comprised γ-secretase relative to PS2-comprised γ-secretase). A compound identified in a method of the invention can be produced using standard organic synthesis techniques as are known to those of skill in the art.

The invention also provides pharmaceutical compositions comprising a binding agent of the invention, methods of treating Alzheimer's disease using such binding agents, and methods of selectively inhibiting PS1-comprised γ-secretase relative to PS2- comprised γ-secretase using such binding agents.

In one aspect, the invention provides a compound that preferentially inhibits Presenilin-1-comprised γ-secretase relative to Presenilin-2-comprised γ-secretase. In one embodiment, the invention comprises a compound that preferentially inhibits Presenilin-1-comprised γ-secretase relative to Presenilin-2-comprised γ-secretase by specifically binding to PS1. Preferably, the compound binds to the N-terminal portion of PS1, most preferably to at least a portion of the N-terminal 1-127 amino acids of PS1.

In certain embodiments, the invention provides methods for identifying compounds that can preferentially inhibit PS1. In one embodiment, the methods comprise: separately incubating a test compound with a first transfected double-knockout cell (hereafter, “first cell type”) expressing Presenilin-1 but not expressing Presenilin-2, and a second transfected double-knockout cell (hereafter, “second cell type”) expressing Presenilin-2 but not expressing Presenilin-1; determining the amount of Aβ1-x (wherein Aβ1-x represents any Aβ peptides longer than Aβ1-23, including Aβ38, Aβ40, and Aβ42) in each cell line; using the amount of Aβ1-x in each cell line to calculate an EC₅₀; and identifying a compound that preferentially inhibits Presenilin-1-comprised γ-secretase relative to Presenilin-2-comprised γ-secretase. A compound of the invention preferentially inhibits Presenilin-1-comprised γ-secretase relative to Presenilin-2-comprised γ-secretase when the EC₅₀ value calculated for the first cell type is smaller than the EC₅₀ value calculated for the second cell type. Preferably a compound of the invention inhibits PS1 relative to PS2 by at least three- to five-fold. Even more preferably, the compound inhibits PS1 relative to PS2 by five-to ten-fold. Even more preferably, the compound inhibits PS1 relative to PS2 by ten- to fifteen-fold, and yet more preferably, fifteen- to twenty-fold. Yet even more preferably, the compound inhibits PS1relative to PS2 by more than twenty-fold. The method can also be used in the same manner to identify antibodies of the invention that preferentially inhibit PS1 activity relative to PS2 activity, wherein the antibodies to be tested are used in place of the test compounds.

In other embodiments, compounds and antibodies that inhibit PS1 can be identified using presenilin chimeras as described in the Examples below. In a particular embodiment, the methods comprise: contacting a presenilin chimera constructed with an N terminal portion of PS1 with a test compound or antibody, and measuring the relative activity of said chimera. A non-limiting example of the method is described below in Examples 1-3. The N terminal portion of PS1 can be the amino acid sequence as shown in SEQ ID NO: 7 (amino acids 1-70 of PS1), SEQ ID NO: 8 (amino acids 1-127 of PS1), or any portion of SEQ ID NO: 7 or SEQ ID NO: 8.

C. Methods for Identifying PS1 Specific Binding Agents

Any type of assay known in the art that can determine the amount of Aβ40 and/or Aβ42 in a cell may be used to determine whether a compound binds PS1 (in particular, the N terminus of PS1) particularly, relative to PS2. In one embodiment the assay is any type of binding assay, preferably an immunological binding assay. Such immunological binding assays are well known in the art (see for example, Asai, ed., Methods in Cell Biology, Vol. 37, Antibodies in Cell Biology, Academic Press, Inc., New York (1993)). Immunological binding assays typically utilize a capture agent to bind specifically to and often immobilize the analyte target antigen. The capture agent is a moiety that specifically binds to the analyte. In one embodiment of the present invention, the capture agent is an antibody or fragment thereof that specifically binds Aβ. The capture agent is an antibody or fragment thereof that specifically binds to an epitope located in the forty amino acid residues of Aβ. In a preferred embodiment, the capture agent is an antibody or fragment thereof that specifically binds to an epitope located in the first 23 amino acid residues of Aβ (i.e., Aβ1-23).

Immunological binding assays frequently utilize a labeling agent that will signal the existence of the bound complex formed by the capture agent and antigen. The labeling agent can be one of the molecules comprising the bound complex; i.e. it can be labeled specific binding agent or a labeled anti-specific binding agent antibody. Alternatively, the labeling agent can be a third molecule, commonly another antibody, which binds to the bound complex. The labeling agent can be, for example, an anti-specific binding agent antibody bearing a label. The second antibody, specific for the bound complex, may lack a label, but can be bound by a fourth molecule specific to the species of antibodies which the second antibody is a member of. For example, the second antibody can be modified with a detectable moiety, such as biotin, which can then be bound by a fourth molecule, such as enzyme-labeled streptavidin. Other proteins capable of specifically binding immunoglobulin constant regions, such as protein A or protein G may also be used as the labeling agent. These binding proteins are normal constituents of the cell walls of streptococcal bacteria and exhibit a strong non-immunogenic reactivity with immunoglobulin constant regions from a variety of species (see, for example, Akerstrom, J Immunol, 135:2589-2542 (1985); and Chaubert, Mod Pathol, 10:585-591 (1997)). In one embodiment of the present invention, the labeling agent comprises an antibody or fragment thereof that specifically binds the first twenty-three amino acid residues of Aβ (Aβ1-23). In a preferred embodiment, the labeling agent comprises an antibody or fragment thereof that specifically binds to an epitope located in the first 3 amino acid residues of Aβ (i.e., Aβ1-3). In one embodiment of the present invention, the labeling agent comprises an antibody or fragment thereof that specifically binds the first twenty-three amino acid residues of Aβ (Aβ1-23). In a preferred embodiment, the labeling agent comprises an antibody or fragment thereof that specifically binds to an epitope located in the first 3 amino acid residues of Aβ (i.e., Aβ1-3).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures.

Assays that demonstrate inhibition of γ-secretase-mediated cleavage of APP can utilize any of the known forms of APP, including the non-limiting examples of the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APPswe), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the gamma-secretase cleavage site, wild type (WT) or mutated form, e.g., APPswe, as described, for example, in U.S. Pat. Nos. 5,441,870, 5,605,811, 5,721,130, 6,018,024, 5,604,102, 5,612,486, 5,850,003, and 6,245,964.

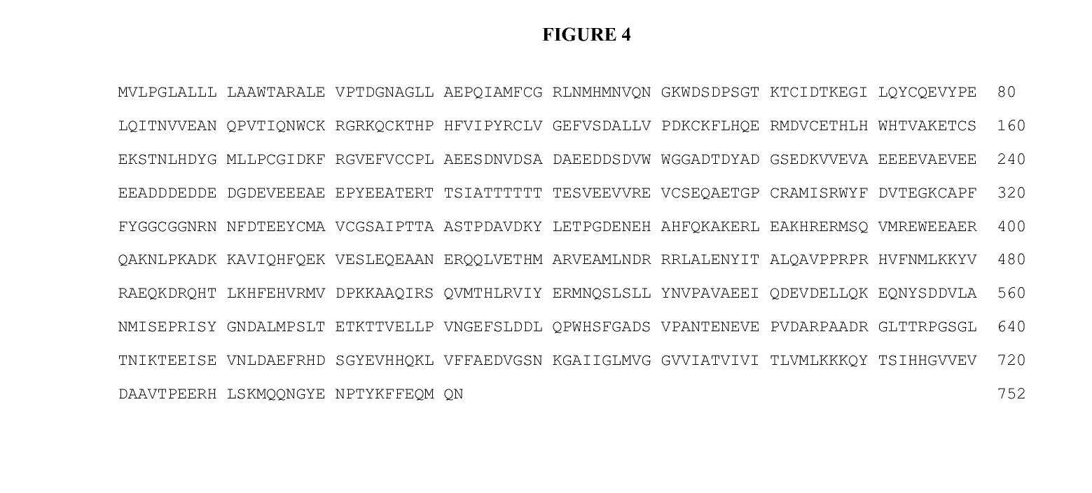

In certain embodiments a cDNA encoding for a form of APP can be transfected into a cell line by the high efficiency transfection methods disclosed herein for producing Presenilin-1 and/or Presenilin-2 knockout fibroblasts. Briefly, high efficiency transfection of Presenilin-1/Presenilin-2 knockout fibroblasts can be achieved by introducing APPswe cDNA (e.g., a cDNA encoding the protein of SEQ ID NO:6 in FIG. 4) and either Presenilin-1 cDNA or Presenilin-2 cDNA by electroporation (Amaxa, Inc., Gaithersburg, Md.), or by using GenePorter 2 (Gene Therapy Systems, Inc., San Diego, Calif.), either together or sequentially. Presenilin-1/Presenilin-2 knockout fibroblasts expressing either Presenilin-1 or Presenilin-2 can then be used to identify compounds that preferentially inhibit Presenilin-1-comprised gamma-secretase relative to Presenilin-2-comprised gamma-secretase. See also, Mullan et al., Nature Genetics (1992); 1:345-347), which discloses the sequence of APPswe, and is hereby incorporated by reference in its entirety.

1. Non-Competitive Binding Assays:

Immunological binding assays can be of the non-competitive type. These assays have an amount of captured analyte that is directly measured. For example, in one preferred “sandwich” assay, the capture agent (antibody) can be bound directly to a solid substrate where it is immobilized. These immobilized antibodies then capture (bind to) antigen present in the test sample. The protein thus immobilized is then bound to a labeling agent, such as a second antibody having a label. In another contemplated “sandwich” assay, the second antibody lacks a label, but can be bound by a labeled antibody specific for antibodies of the species from which the second antibody is derived. The second antibody also can be modified with a detectable moiety, such as biotin, to which a third labeled molecule can specifically bind, such as streptavidin. (See, Harlow and Lane, Antibodies, A Laboratory Manual, Ch 14, Cold Spring Harbor Laboratory, NY (1988), incorporated herein by reference in its entirety).

2. Competitive Binding Assays:

Immunological binding assays can be of the competitive type. The amount of analyte present in the sample is measured indirectly by measuring the amount of an added analyte displaced, or competed away, from a capture agent by the analyte present in the sample. In one preferred competitive binding assay, a known amount of analyte, usually labeled, is added to the sample and the sample is then contacted with an antibody (the capture agent). The amount of labeled analyte bound to the antibody is inversely proportional to the concentration of analyte present in the sample. (See, Harlow and Lane, Antibodies, A Laboratory Manual, Ch 14, pp. 579-583, supra).

In another contemplated competitive binding assay, the antibody is immobilized on a solid substrate. The amount of protein bound to the antibody may be determined either by measuring the amount of protein present in a protein/antibody complex, or alternatively by measuring the amount of remaining uncomplexed protein. The amount of protein may be detected by providing a labeled protein. See, Harlow and Lane, Antibodies, A Laboratory Manual, Ch 14, supra).

In yet another contemplated competitive binding assay, hapten inhibition is utilized. Here, a known analyte is immobilized on a solid substrate. A known amount of antibody is added to the sample, and the sample is contacted with the immobilized analyte. The amount of antibody bound to the immobilized analyte is inversely proportional to the amount of analyte present in the sample. The amount of immobilized antibody may be detected by detecting either the immobilized fraction of antibody or the fraction that remains in solution. Detection may be direct where the antibody is labeled or indirect by the subsequent addition of a labeled moiety that specifically binds to the antibody as described above.

3. Utilization of Competitive Binding Assays:

The competitive binding assays can be used for cross-reactivity determinations to permit a skilled artisan to determine if a protein or enzyme complex that is recognized by a specific binding agent of the invention is the desired protein and not a cross-reacting molecule, or to determine whether the antibody is specific for the antigen and does not bind unrelated antigens. In assays of this type, antigen can be immobilized to a solid support and an unknown protein mixture is added to the assay, which will compete with the binding of the specific binding agents to the immobilized protein. The competing molecule also binds one or more antigens unrelated to the antigen. The ability of the proteins to compete with the binding of the specific binding agents/antibodies to the immobilized antigen is compared to the binding by the same protein that was immobilized to the solid support to determine the cross-reactivity of the protein mix.

4. Other Binding Assays:

Other non-immunologic techniques for detecting Aβ and Aβ fragments that do not require the use of Aβ specific antibodies may also be employed. For example, two-dimensional gel electrophoresis may be employed to separate closely related soluble proteins present in a fluid sample. Antibodies that are cross-reactive with many fragments of APP, including Aβ, may then be used to probe the gels, with the presence of Aβ being identified based on its precise position on the gel. In the case of cultured cells, the cellular proteins may be metabolically labeled and separated by SDS-polyacrylamide gel electrophoresis, optionally employing immunoprecipitation as an initial separation step.

The present invention also provides Western blot methods to detect or quantify the presence of Aβ in a sample. The technique generally comprises separating sample proteins by gel electrophoresis on the basis of molecular weight and transferring the proteins to a suitable solid support, such as nitrocellulose filter, a nylon filter, or derivatized nylon filter. The sample is incubated with antibodies or fragments thereof that specifically bind Aβ and the resulting complex is detected. These antibodies may be directly labeled or alternatively may be subsequently detected using labeled antibodies that specifically bind to the antibody.

D. Assays for Determining Efficacy of PS1 Specific Binding Agent

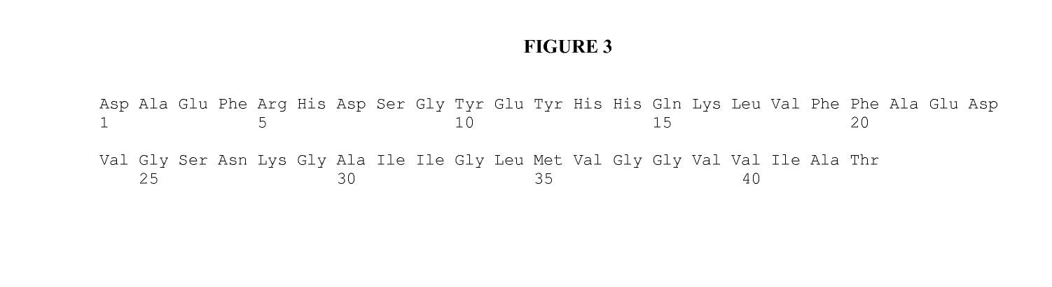

In one embodiment, the methods of the invention comprise a specific binding agent to Aβ. In a preferred embodiment the method comprises at least one antibody to Aβ, and more preferably at least two antibodies to Aβ. When the method comprises at least two antibodies to Aβ, one antibody preferably acts as a “capture” molecule, while the other antibody acts as the detection or “labeled” molecule. In certain embodiments the capture antibody can recognize an epitope of Aβ, which is located in the N-terminal portion of the amino acid sequence (see, FIG. 3). More particularly, the capture antibody preferably recognizes an epitope within amino acids 1-23 of Aβ.

Products characteristic of APP cleavage can be measured by immunoassay using various antibodies such as those as described, for example, in Pirttila et al., 1999, Neuro. Lett. 249:21-4, and in U.S. Pat. No. 5,612,486 (both incorporated by reference in their entireties). Useful antibodies to detect Aβ include, for example, the monoclonal antibody 6E10 (Senetek, St. Louis, Mo.) that specifically recognizes an epitope on amino acids 1-16 of the Aβ peptide; antibodies 162 and 164 (New York State Institute for Basic Research, Staten Island, N.Y.) that are specific for human Aβ1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

E. Antibody Preparation

In certain embodiments, the invention provides antibodies that bind to the N-terminal portion of PS1. The antibodies of the invention can be produced using conventional techniques as described herein. Suitable antigens (also referred to herein as “immunogens”) for producing an antibody of the invention are described above.

Antibodies specific for Aβ may be prepared against a suitable antigen or hapten comprising the desired target epitope, such as the junction region consisting of amino acid residues 13-28, the C-terminus consisting of about amino acid residues 29-42 or 43, and the amino terminus consisting of amino acid residues 1-16.

Conveniently, synthetic peptides for preparing antibodies may be prepared by conventional solid phase techniques, coupled to a suitable immunogen, and used to prepare antisera or monoclonal antibodies by conventional techniques. Suitable peptide haptens will usually comprise at least five contiguous residues within Aβ and may include more than six residues.

Synthetic polypeptide haptens may be produced by the well-known Merrifield solid-phase synthesis technique in which amino acids are sequentially added to a growing chain (Merrifield (1963) J. Am. Chem. Soc. 85:2149-2156). The amino acid sequences may be based on the sequence of βAP set forth above.

Once a sufficient quantity of polypeptide hapten has been obtained, it may be conjugated to a suitable immunogenic carrier, such as serum albumin, keyhole limpet hemocyanin, or other suitable protein carriers, as generally described in Hudson and Hay, Practical Immunology, Blackwell Scientific Publications, Oxford, Chapter 1.3, 1980, the disclosure of which is incorporated herein by reference. An exemplary immunogenic carrier that has been useful is αCD3κ antibody (Boehringer-Mannheim, Clone No. 145-2C11).

Once a sufficient quantity of the immunogen has been obtained, antibodies specific for the desired epitope may be produced by in vitro or in vivo techniques. In vitro techniques involve exposure of lymphocytes to the immunogens, while in vivo techniques require the injection of the immunogens into a suitable vertebrate host. Suitable vertebrate hosts are non-human, including mice, rats, rabbits, sheep, goats, and the like. Immunogens are injected into the animal according to a predetermined schedule, and the animals are periodically bled, with successive bleeds having improved titer and specificity. The injections may be made intramuscularly, intraperitoneally, subcutaneously, or the like, and an adjuvant, such as incomplete Freund's adjuvant, may be employed.

If desired, monoclonal antibodies can be obtained by preparing immortalized cell lines capable of producing antibodies having desired specificity. Such immortalized cell lines may be produced in a variety of ways. Conveniently, a small vertebrate, such as a mouse is hyperimmunized with the desired immunogen by the method just described. The vertebrate is then killed, usually several days after the final immunization, the spleen cells removed, and the spleen cells immortalized. The manner of immortalization is not critical. Monoclonal antibodies useful in the invention may be made by the hybridoma method as described in Kohler et al., Nature 256:495 (1975); the human B-cell hybridoma technique (Kosbor et al., Immunol Today 4:72 (1983); Cote et al., Proc Natl Acad Sci (USA) 80:2026-2030 (1983); Brodeur et al., Monoclonal Antibody Production Techniques and Applications, pp. 51-63, Marcel Dekker, Inc., New York, (1987)) and the EBV-hybridoma technique (Cole et al., Monoclonal Antibodies and Cancer Therapy, Alan R Liss Inc, New York N.Y., pp 77-96, (1985)).

When the hybridoma technique is employed, myeloma cell lines can be used. Such cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). For example, cell lines used in mouse fusions are Sp-20, P3-X63/Ag8, P3-X63-Ag8.653, NS1/1.Ag 41, Sp210-Ag14, FO, NSO/U, MPC-11, MPC11-X45-GTG 1.7 and S194/5XX0 Bul; cell lines used in rat fusions are R210.RCY3, Y3-Ag 1.2.3, IR983F and 4B210. Other cell lines useful for cell fusions are U-266, GM1500-GRG2, LICR-LON-HMy2 and UC729-6. Hybridomas and other cell lines that produce monoclonal antibodies are contemplated to be novel compositions of the present invention.

The phage display technique may also be used to generate monoclonal antibodies from any species. Preferably, this technique is used to produce fully human monoclonal antibodies in which a polynucleotide encoding a single Fab or Fv antibody fragment is expressed on the surface of a phage particle. (Hoogenboom et al., J Mol Biol 227: 381 (1991); Marks et al., J Mol Biol 222: 581 (1991); see also U.S. Pat. No. 5,885,793)). Eachphage can be “screened” using binding assays described herein to identify those antibody fragments having affinity for Aβ. Thus, these processes mimic immune selection through the display of antibody fragment repertoires on the surface of filamentous bacteriophage, and subsequent selection of phage by their binding to Aβ. One such procedure is described in PCT Application No. PCT/US98/17364, filed in the name of Adams et al., which describes the isolation of high affinity and functional agonistic antibody fragments for MPL- and msk-receptors using such an approach. In this approach, a complete repertoire of human antibody genes can be created by cloning naturally rearranged human V genes from peripheral blood lymphocytes as previously described (Mullinax et al., Proc Natl Acad Sci (USA) 87: 8095-8099 (1990)). Specific techniques for preparing monoclonal antibodies are described in Antibodies: A Laboratory Manual, Harlow and Lane, eds., Cold Spring Harbor Laboratory, 1988, the full disclosure of which is incorporated herein by reference.

In addition to monoclonal antibodies and polyclonal antibodies (antisera), the detection techniques of the present invention will also be able to use antibody fragments, such as F(ab), Fv, V_{L}, V_{H}, and other fragments. In the use of polyclonal antibodies, however, it may be necessary to adsorb the anti-sera against the target epitopes in order to produce a monospecific antibody population. It will also be possible to employ recombinantly produced antibodies (immunoglobulins) and variations thereof as now well described in the patent and scientific literature. See, for example, EPO 8430268.0; EPO 85102665.8; EPO 85305604.2; PCT/GB 85/00392; EPO 85115311.4; PCT/US86/002269; and Japanese application 85239543, the disclosures of which are incorporated herein by reference. It would also be possible to prepare other recombinant proteins that would mimic the binding specificity of antibodies prepared as just described.

F. Generation of Knockout Cells

The cell types that can be used with the invention include any type of cell, either naturally occurring or artificially constructed, that express Presenilin-1 and not Presenilin-2, or express Presenilin-2 and not Presenilin-1. In one embodiment, the cell types are constructed from cells that comprise Presenilin-1 and Presenilin-2 double knockout genotype. Using known methods, or those disclosed herein, one of skill in the art can transform/transfect such double knockout cells with a cDNA encoding for either Presenilin-1 or Presenilin-2 and construct cell types that express Presenilin-1 and not Presenilin-2, or express Presenilin-2 and not Presenilin-1, as well as a cDNA encoding a γ-secretase substrate, either sequentially or at the same time. Any known methods of recombinant nucleic acid technology, genetic manipulation (i.e., creating knockout strains), and cell transformation/transfection can be used, as well as those methods as described in detail herein.

In certain embodiments of the invention, the PS1/PS2 knockout cells are made as described in An Herreman et al, “Total inactivation of gamma-secretase activity in presenilin-deficient embryonic stem cells.” Nature Cell Biology 2, 461-462 (2000), which is hereby incorporated by reference in its entirety. Mouse fibroblasts are derived from the knockout cell lines as described in An Herreman et al., “Presenilin 2 deficiency causes a mild pulmonary phenotype and no changes in amyloid precursor protein processing but enhances the embryonic lethal phenotype of presenilin 1 deficiency”, PNAS 1999; 96: 11872-11877, which is herein incorporated by reference in its entirety. Generation of knockout cell lines is known by those of skill in the art, and is described, for example, in U.S. patent application Ser. No. 10/082,804, which is hereby incorporated by reference in its entirety. In preferred embodiments of the invention, the first cell type is a Presenilin-1/Presenilin-2 double knockout cell line transfected with a vector comprising Presenilin-1 cDNA and the second cell type is a Presenilin-1/Presenilin-2 double knockout cell line transfected with a vector comprising Presenilin-2. One appropriate vector, and the vector chosen for the exemplary embodiments detailed in the Examples is pCF, which was modified with pcDNA3 (Invitrogen, Calif., USA) by inserting the adenoviral tripartite leader sequence (see, Berkner, K. L., et al., J. Virol. (1987) 61:1213-1220) between the CMV promoter and the EcoR1 site.

In other aspects the invention provides compounds that preferentially inhibit Presenilin-1-comprised γ-secretase relative to Presenilin-2-comprised γ-secretase, pharmaceutical compositions comprising such compounds, methods of treating Alzheimer's disease using such compounds, and methods of selectively inhibiting PS1-comprised γ-secretase relative to PS2-comprised γ-secretase using such compounds.

Thus, in one aspect the invention relates to a compound that preferentially inhibits Presenilin-1-comprised γ-secretase relative to Presenilin-2-comprised γ-secretase. In an embodiment, a compound that preferentially inhibits Presenilin-1-comprised γ-secretase relative to Presenilin-2-comprised γ-secretase is identified by the assay method of the invention, for example, by separately incubating a compound with a first transfected double-knockout cell (hereafter, “first cell type”) expressing Presenilin-1 but not expressing Presenilin-2, and a second transfected double-knockout cell (hereafter, “second cell type”) expressing Presenilin-2 but not expressing Presenilin-1; determining the amount of Aβ1-x in each cell line; using the amount of Aβ1-x in each cell line to calculate an EC₅₀; and identifying a compound that preferentially inhibits Presenilin-1-comprised γ-secretase relative to Presenilin-2-comprised γ-secretase. In an embodiment, a compound of the invention preferentially inhibits Presenilin-1-comprised γ-secretase relative to Presenilin-2-comprised γ-secretase when the EC₅₀ value calculated for the first cell type is smaller than the EC₅₀ value calculated for the second cell type. Preferably a compound of the invention inhibits PS1relative to PS2 by at least three- to five-fold. Even more preferably, the compound inhibits PS1 relative to PS2 by five-to ten-fold. Even more preferably, the compound inhibits PS1relative to PS2 by ten- to fifteen-fold, and yet more preferably, fifteen- to twenty-fold. Yet even more preferably, the compound inhibits PS1 relative to PS2 by more than twenty-fold.

In another embodiment, a compound of the invention comprises a sulfonamide functional group. In a preferred embodiment a compound of the invention is selected from the sulfonamide series of γ-secretase inhibitors. Thus, in various embodiments the invention provides for identification of a novel compound that preferentially inhibits PS1-comprised γ-secretase relative to PS2-comprised γ-secretase and/or identification of a known compound for a novel use (i.e., preferential inhibition of PS1-comprised γ-secretase relative to PS2-comprised γ-secretase). Any such compound can be either purchased from a commercial source and/or produced using standard organic synthesis techniques as are known to those of skill in the art.

G. Methods of Treatment

In certain embodiments, the invention provides compositions comprising the above-described specific binding agents, in combination with a pharmaceutically acceptable salt, vehicle, carrier, diluent, and/or adjuvant.

The compositions of the invention can be administered orally, enterally, parenterally, (IV, IM, depo-IM, SQ, and depo SQ), sublingually, intranasally (inhalation), intrathecally, topically, or rectally. Dosage forms known to those of skill in the art are suitable for delivery of the specific binding agents of the invention.

Compositions are provided that contain therapeutically effective amounts of the specific binding agents of the invention. The specific binding agents are preferably formulated into suitable pharmaceutical preparations such as tablets, capsules, or elixirs for oral administration or in sterile solutions or suspensions for parenteral administration. Typically the specific binding agents described above are formulated into pharmaceutical compositions using techniques and procedures well known in the art.

About 1 to 500 mg of a compound or mixture of specific binding agents of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more specific binding agents of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the specific binding agents provided herein include any such carriers known to those skilled in the art to be suitable for the particular mode of administration. In addition, the active materials can also be mixed with other active materials that do not impair the desired action, or with materials that supplement the desired action, or have another action. The specific binding agents may be formulated as the sole pharmaceutically active ingredient in the composition or may be combined with other active ingredients.

Where the specific binding agents exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the specific binding agents, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The specific binding agents of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the specific binding agents in known in vitro and in vivo model systems for the treated disorder.

The specific binding agents and compositions of the invention can be enclosed in multiple or single dose containers. The enclosed specific binding agents and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active specific-binding agent or specific binding agents can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or specific binding agents of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The specific binding agents can also be administered as a component of an elixir, suspension, syrup, wafer, chewing gum or the like. A syrup may contain, in addition to the active specific binding agents, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active specific binding agents may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, specific binding agents of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the specific binding agents of the invention need to be administered only once or twice daily.

The oral dosage forms can be administered to the subject 1, 2, 3, or 4 times daily. It is preferred that the specific binding agents of the invention be administered either three or fewer times, more preferably once or twice daily. Hence, it is preferred that the specific binding agents of the invention be administered in oral dosage form. It is preferred that whatever oral dosage form is used, that it be designed so as to protect the specific binding agents of the invention from the acidic environment of the stomach. Enteric coated tablets are well known to those skilled in the art. In addition, capsules filled with small spheres each coated to protect from the acidic stomach, are also well known to those skilled in the art.

As noted above, depending on whether asymmetric carbon atoms are present, the specific binding agents of the invention can be present as mixtures of isomers, as racemates, or in the form of pure isomers.

Salts of specific binding agents are preferably the pharmaceutically acceptable or non-toxic salts. For synthetic and purification purposes it is also possible to use pharmaceutically unacceptable salts.

In certain embodiments, the composition can comprise an additional agent effective for the treatment of Alzheimer's disease, as are known in the art.

In one aspect, the invention provides methods of treating and/or preventing Alzheimer's disease in a subject in need of such treatment, comprising administering to the subject an effective amount of a compound, or salt thereof, identified by the assay method of the invention. In one aspect, this method of treatment can be used where the subject is diagnosed with Alzheimer's disease. In another aspect, this method of treatment can help prevent or delay the onset of Alzheimer's disease. In another aspect, this method of treatment can help slow the progression of Alzheimer's disease. In another aspect, this method of treatment can prevent a disease, such as those listed above, from developing or progressing.

In an embodiment of this aspect, the effective amount of a compound discovered by the assay method of the invention is contained in a composition comprising a pharmaceutically acceptable salt, carrier, vehicle, adjuvant, or diluent.

In a preferred aspect of the methods of the invention, the subject is human.

The methods of treatment employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg. In a preferred aspect, the therapeutically effective amounts for oral administration is from about 1 mg/day to about 100 mg/day; and for parenteral administration from about 5 to about 50 mg daily. In a more preferred aspect, the therapeutically effective amounts for oral administration is from about 5 mg/day to about 50 mg/day.

In another embodiment, the invention provides a method of selectively inhibiting Presenilin-1-comprised γ-secretase relative to Presenilin-2-comprised γ-secretase in a cell, comprising contacting a cell with a compound identified by the assay of the invention effective to selectively inhibit Presenilin-1-comprised γ-secretase relative to Presenilin-2-comprised γ-secretase. In an embodiment the method inhibits Presenilin-1-comprised γ-secretase by about three- to five-fold relative to Presenilin-2-comprised γ-secretase. Even more preferably, the method inhibits PS1 relative to PS2 by about five-fold to about ten-fold, more preferably by about ten-fold to fifteen-fold, and yet more preferably, by about fifteen-fold to about twenty-fold. Yet even more preferably, the method inhibits PS1 relative to PS2 by more than about twenty-fold.

In one embodiment, the cell is a mammalian cell. In a preferred embodiment the cell is a human cell. In other embodiments the cell is an isolated mammalian cell, preferably an isolated human cell.

In an embodiment this method of selectively inhibiting Presenilin-1-comprised γ-secretase relative to Presenilin-2-comprised γ-secretase can be used to treat a subject that has a disease or a disorder related to activity of Presenilin-1-comprised γ-secretase. In one embodiment, the subject demonstrates clinical signs of a disease or a disorder related to Presenilin-1-comprised γ-secretase. In another embodiment, the subject is diagnosed with a disease or a disorder related to Presenilin-1-comprised γ-secretase. In a preferred embodiment the disease or disorder relates to Presenilin-1-comprised γ-secretase and not to Presenilin-2-comprised γ-secretase. As the specific binding agents useful in this method are identified by the assay of the invention as selective inhibitors of Presenilin-1-comprised γ-secretase relative to Presenilin-2-comprised γ-secretase methods of treating disorders or diseases related to Presenilin-1-comprised γ-secretase can be treated without adversely effecting Presenilin-2-comprised γ-secretase activity (e.g., such as Notch signaling).

The Examples that follow are merely illustrative of specific embodiments of the invention, and are not to be taken as limiting the invention, which is defined by the appended claims.

### EXAMPLES

### Example 1

### Identification of Structural Elements Responsible for Differential Aβ Production by PS1 and PS2

We found that PS1-transfected double KO cells produce several times more total Aβ (Aβ40+Aβ42) than PS2-transfected cells. Up to 38-fold differences were reported by others when comparing PS1 and PS2 single knockout cells, See Lai, et al., J. Biol. Chem., June 2003; 278: 22475-22481. In order to understand the basis for this difference in Aβ production we identified the specific structural elements in PS1 and PS2 that conferred Aβ-producing activity in each.

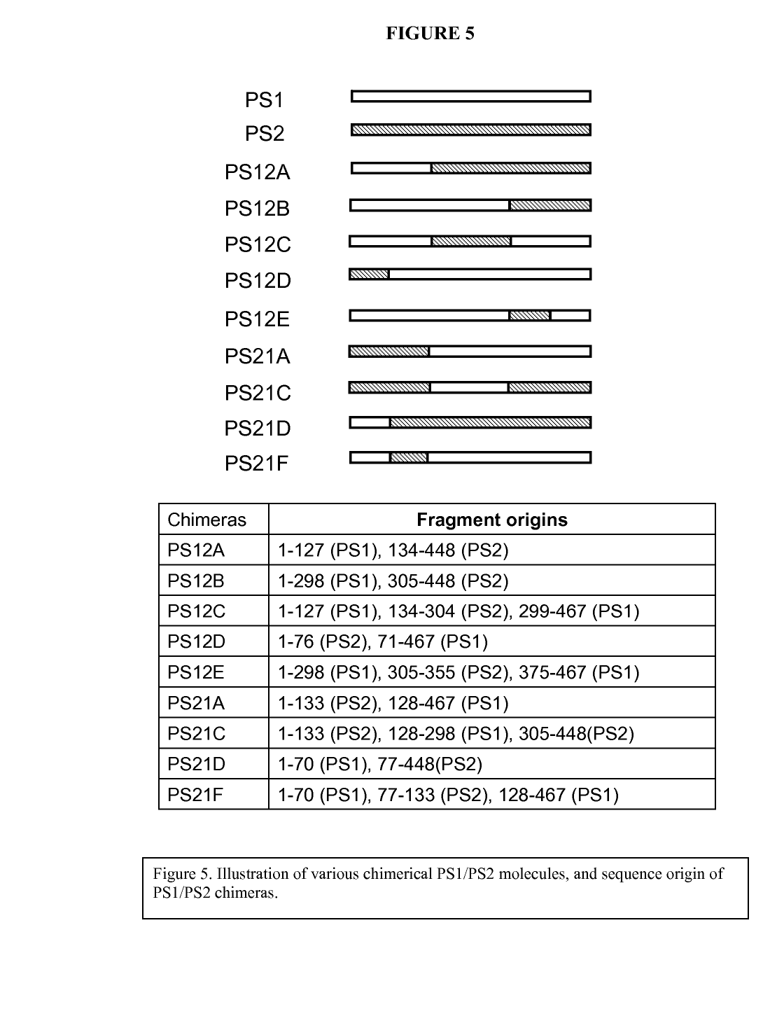

To look for structural elements that determine total Aβ levels, we prepared various chimeric presenilin molecules derived from portions of PS1 and PS2, and subcloned them into the pCF vector. The various chimeric molecules are illustrated in FIG. 5, and sequence origin of PS1 or PS2 portions are also shown in FIG. 5.

Transient transfection was then performed on the PS1/PS2 double knockout cells with APPsw plus either PS1, or PS2, or a chimeric molecule (as indicated in FIG. 5). Aβ1-x levels were determined in conditioned medium from cells of each transfection. Methods for generation of PS1 and PS2 knockout cells types, as well as the transfection of PS1, PS2, or chimeras, are described above.

Molecular Cloning and Construction of Chimeras

Human PS1, PS2, and APPsw cDNA inserts were subcloned into pCF vector, which was modified with pcDNA3 (Invitrogen, CA, USA) by inserting the adenoviral tripartite leader sequence (Berkner et al, (1987) J. Virol. April; 61(4): 1213-20. Abundant expression of polyomavirus middle T antigen and dihydrofolate reductase in an adenovirus recombinant) 38 bp upstream of the starting ATG codon, between the CMV promoter and the EcoR1 site. Construction of presenilin chimeras was PCR-based. For making chimeras that contain PS1backbone and a PS2 fragment, we first generated a large PCR fragment that contained the entire pCF vector plus all PS1 sequence to be retained, and a small PCR fragment that only contained the PS2 fragment to be used in the final chimera. The two PCR fragments were then ligated in a blunt-end fashion by Rapid DNA ligation kit (Roche, Ind., USA). We used pfu Turbo DNA polymerase kit (Strategene, Calif., USA) for all PCR reactions. To avoid potential mutations introduced by PCR, we first sequenced the entire insert in both strands. We then excised the sequence-verified insert from the PCR-generated vector, and subcloned it into another pCF vector that did not go through PCR. For making chimeras that contained PS2 backbone and a PS1 fragment, we first generated a large PCR fragment that contained the entire pCF vector plus all PS2 sequence to be retained, and a small PCR fragment that only contained the PS1 fragment to be used in the final chimera. All other cloning procedures were the same as described above.

### Example 2

### Generation of a Standard Curve

Since differences in Aβ levels may be due to either a difference in presenilin activity, or presenilin expression level, we needed to find out relative expression level of different presenilin molecules, and then normalize Aβ levels by the relative protein level. The normalized Aβ levels should reflect relative activity, or enzyme turnover rate, of different presenilin constructs.

However, determination of relative expression levels of different chimeras was not a straightforward task, mainly because no single PS1 or PS2 antibody can detect both PS1 and PS2, as well as all the chimeras. For example, although signals on western blots generated by Mab1563 (Chemicon, Temecula, Calif., USA) for PS1N-terminus, and signals by PC235T (Oncogene, San Diego, Calif., USA) for PS2 C-terminus are readily detectable, the signals from the two antibodies can not be compared to determine the relative expression level of PS1 and PS2 proteins due to intrinsic differences in antibody properties, e.g. affinity. This presented a problem in determining the correlation between signals generated by the PS1 and PS2 antibodies for their respective antigens.

This problem was solved by focusing on PS12B, a presenilin chimeric molecule, in which the N-terminus is from PS1 and C-terminus is from PS2. PS12B is first synthesized as a single polypeptide chain and subsequently is cleaved into a mature PS1 N-terminus which is recognized by Mab1563, and a mature PS2 C-terminus which is recognized by PC235T. Because both NTF (N-terminal fragment-PS1 epitope) and CTF (C-terminal fragment, PS2 epitope) are derived from the same polypeptide chain, there should be a fixed ratio between the two fragments. Assuming that the NTF and CTF have the same stability in cells, the ratio will be 1:1, which implies that the NTF and CTF are present in equal molar concentrations in the cells. Therefore, when both Mab1563- and PC235T-detected bands on a Western are of similar intensity, it can be concluded that the two antibodies, under the particular experimental conditions, have similar sensitivity for the two different antigens, and the signals can be compared.

Since it is not always practical to obtain identical signals for PS1 and PS2 antibodies on a Western blot, in practice, gels were loaded with different amount of PS12B, and both Mab1563 and PC235T signals were detected on the same blots. The Western signals from PS12B can be used to establish standard curves to derive the relative amount of other chimeras, or PS1, or PS2.

### Example 3

### Comparison of Expression Levels

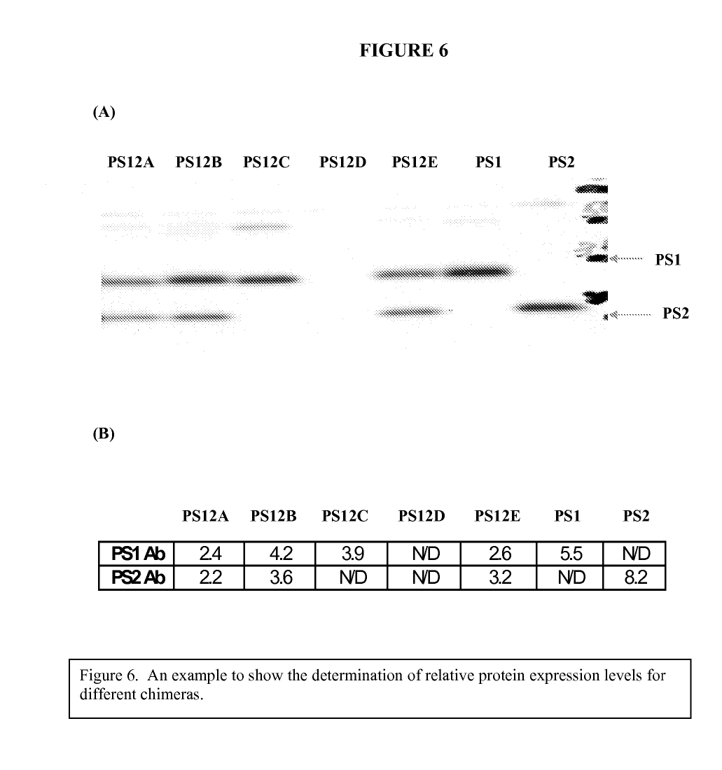

With the standard curves, one can compare relative expression levels of different chimeras, with samples loaded on the same Western gel as the PS12B standards. FIG. 6 shows an example of how relative protein expression levels were determined for different chimeras. In the experiment, each presenilin cDNA construct was co-transfected with APPsw into the double KO cells. After overnight incubation, cells were lysed, and proteins were extracted from the cells for each transfection. For Western analysis, 5 μg protein preparations were loaded, and presenilin NTF and CTF were detected with MAB1563 and PC235T on the same blot (various amount of PS12B were loaded on the same gel as standards, but not shown here for clarity of display). Western signals were first quantitated by scanning films (A), and the signals were then compared to the standard curves for each antibody, and expressed as equivalent amount of protein preparations from PS12B-transfected cells that would generate the same amount of signals on Western.

The methods described in Examples 2-4, below, were used to determine relative activity (measured as Aβ production) of the chimera constructs. Table 1 illustrates the determination of relative activity of the various presenilin chimera constructs shown in FIG. 6. Basically, protein levels determined in FIG. (6B) were normalized by arbitrarily assigning the level of PS2 to 1, which gave the values in the third column in Table 1. Finally, relative activity was derived by first dividing Aβ levels (2^{nd} column in Table 1) with relative protein amount (3^{rd} column in Table 1), and normalized again by assigning the relative activity of PS2 to 1.

Table 1 provides an example to demonstrate the determination of relative activity of various presenilin constructs, by dividing Aβ levels with relative protein amount, and arbitrarily assigning the relative activity of PS2 to 1.

TABLE 1

| cDNA | Aβ (pg/ml) | Relative protein amount | Relative activity |
|---|---|---|---|
| PS1 | 3500 | 0.67 | 8.7 |
| PS2 | 600 | 1.0 | 1.0 |
| PS12A | 1500 | 0.28 | 8.9 |
| PS12B | 1800 | 0.48 | 6.3 |
| PS12C | 2800 | 0.48 | 9.7 |
| PS12E | 1840 | 0.35 | 8.8 |

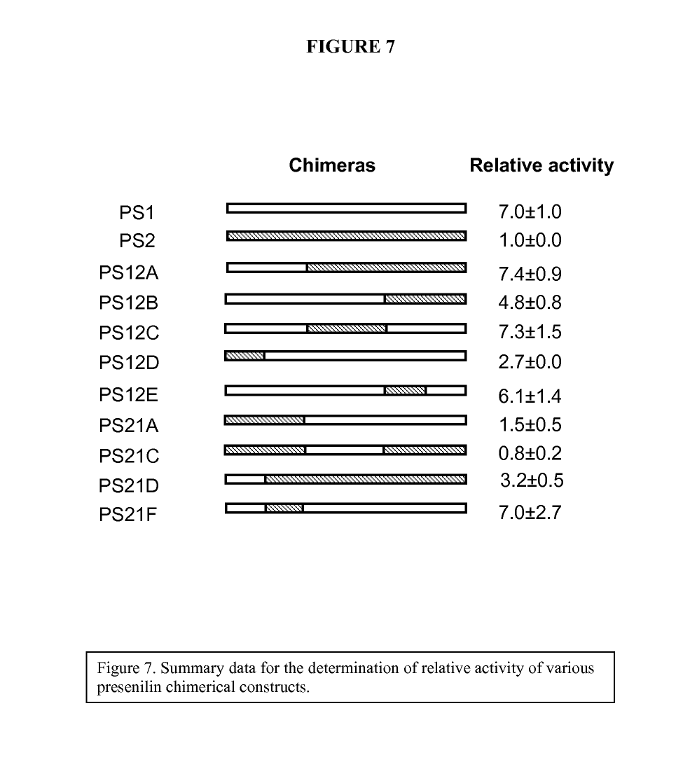

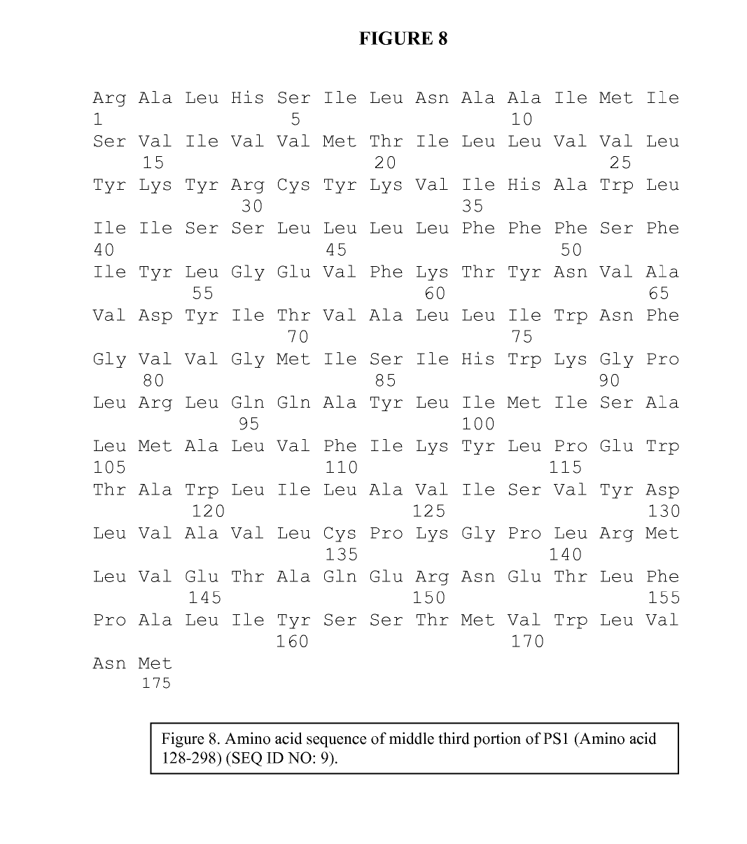

The process of deriving relative activity illustrated above was applied to additional chimeras in other experiments and all the relevant data from several other repeat experiments are summarized in FIG. 7.

It is clear from FIG. 7 that Chimeras PS12A, C, and E all have similar relative activity as PS1, and that PS12B has slightly lower relative activity than PS1, but still much higher than PS2.

FIG. 7 shows that PS12A, PS12B, and PS12C had similar activity as PS1, while PS21A, and PS21C had similar activity as PS2, and PS12D and PS21D are intermediate between PS1 and PS2, thus leading to the conclusion that the N-terminal third of PS1 conferred a high relative activity, with the first half (amino acid residues 1-70 in PS1) to be slightly more important than the second half (amino acid residues 71-127 in PS1) of this region. Although data on PS21F may suggest that the N-terminal sixth accounts for the entire contribution to activity by the N-terminal third, data on PS12D and PS21D contradict this observation. So overall, it is the N-terminal third (amino acid residues 1-127 in PS1) that appear to confer high Aβ or low Aβ γ-secretase activity.

### Example 4

### ELISA assays for A131-x

A131-x represents any Aβ peptides longer than Aβ1-23, including Aβ38, Aβ40, and Aβ42, since Aβ1-x is defined operationally by an ELISA assay using proprietary antibody mAb 266 for capture and proprietary antibody mAb 3D6 for detection. The epitope for mAb266 is Aβ16-23, and the epitope for mAb 3D6 is Aβ1-5. The peptide sequence of Aβ can be found in FIG. 3. Aβ40 ELISA employed antibodies mAb 266 as capture and 2G3 (specific for Ab40) as detection, respectively. Furthermore, Aβ42 ELISA employed antibodies mAb 266 as capture and 21F12 as detection, respectively. Hybridomas producing antibodies against Aβ16-23 were generated by standard murine fusion procedures as detailed in Kohler and Milstein (Nature 256:495 1975) and U.S. Pat. No. 4,666,829 which are hereby incorporated by reference in their entireties. See also “Detailed Description” herein. Briefly, two BALB/c mice immunized with Aβ13-28 conjugated to 2C-11 (a T-cell receptor monoclonal antibody) were sacrificed and the spleens removed. Mixed splenocytes were obtained by pressing the spleens through a 30 mesh stainless steel screen. These were fused with P3X63Ag8 murine myeloma cells (aminopterin sensitive) at a fusion ratio of 10:1 in 35% polyethylene-glycol. These cells were plated out in 96 well tissue culture plates in the presence of 2×10⁶ thymocytes/ml. Hybridomas were selected for by growing the cells in the presence of aminopterin poisoned Dulbecco's modified Eagle's media augmented with hypoxanthine, thymine and 10% fetal bovine serum. Hybridomas were screened for reactivity against Aβ13-28 and AAP protein via ELISA. Positive clones were sub-cloned twice. Aliquots of the clones were frozen and stored in liquid nitrogen. Supernatants from positive clones were produced in large quantities for further purification of monoclonal antibodies. A similar method is used to produce monoclonal antibodies to Aβ1-3, where the mice were originally immunized with Aβ1-5 conjugated to polyclonal sheep anti-mouse antibody.

For ELISA assays, each well of 96-well ELISA plates was coated with 100 μl of 10 μg/ml 266 in Well Coating Buffer (pH 8.5) at 4 degrees overnight, and blocked with 0.25% human BSA solution at 25 degrees for 120 minutes. The plate can be used directly without wash, after removing blocking solution.

ELISA assays were performed at room temperature. Fifty μl of conditioned medium from overnight culture of transfected cells, with or without gamma secretase inhibitors, were added to each well of ELISA plates, and incubated for 1 hour. After washing plates with Tris-buffered saline (TBS) plus 0.05% Tween-20, 50 μl biotinylated 3D6 antibody at 0.5 μg/ml was added to each well and incubate for 45 minutes. Then, plates were washed with Tris-buffered saline (TBS) plus 0.05% Tween-20, and 50 μl streptavidin-HRP conjugate (1 to 5000 dilution, Amersham, Piscataway, N.J., USA, catalogue number: RPN4401) was added to each well and incubated for 30 min. Next, plates were washed with Tris-buffered saline (TBS) plus 0.05% Tween-20, and 50 μl substrate (1-step slow TMB-Elisa, Pierce, Woburn, Mass., USA, catalogue number: 34024) was added to each well and incubated for 15 min. Finally, substrate reactions were terminate by adding to each well 15 ul 2 NH₂SO₄, and OD readings were obtained on SpectraMax Plus (Molecular Devices, Sunnyvale, Calif., USA). The Aβ concentration of samples was then obtained by comparing sample OD readings to those of standards.

EC₅₀ values were derived by curve fitting of Aβ1-x levels, for samples treated with various concentrations of gamma secretase inhibitors, with XLfit software program (IDBS, Alameda, Calif., USA). Differences in EC₅₀ values obtained for Presenilin-1 transfected cells and Presenilin-2 transfected cells exposed to a test compound served as an indicator of differential inhibition.

### Example 5

### Identification of Compounds that Preferentially Inhibit Presenilin-1-Comprised γ-Secretase Relative to Presenilin-2-Comprised γ-Secretase

To identify compounds that preferentially inhibit Presenilin-1-comprised γ-secretase relative to Presenilin-2-comprised γ-secretase, known γ-secretase inhibitor compounds are incubated with both Presenilin-1 transfected cells and Presenilin-2 transfected cells at various concentrations overnight. Transfected mouse fibroblasts derived from the PS1/PS2 double knockout cells are grown at 37 degree under 10% CO₂ in Dulbecco's modified Eagle's medium (DMEM) containing 2-10% fetal bovine serum (FBS) and 100 μg/ml penicillin/streptomycin (Pen/Strp) (Invitrogen Corporation, Carlsbad, Calif., USA).

Cell culture medium is then removed from the transfected cell lines and analyzed for Aβ1-x levels by ELISA assay, as described in Example 1. ELISA assays are performed using ELISA plates coated with the mAb 266 to capture Aβ peptides and then by detecting Aβ peptides with biotinylated mAb 3D6. EC₅₀ values are derived for all of the test compounds. Differences in EC₅₀ values obtained for Presenilin-1 transfected cells and Presenilin-2 transfected cells exposed to a test compound serve as an indicator of differential inhibition.

### Example 6

### Transfection with GenePorter 2

About 30,000 cells were placed into each well of 96-well plates. Twenty hours later, culture medium was replaced with 60 μl Optimem medium (Invitrogen Corporation, Carlsbad, Calif., USA) in each well. Meanwhile, the following 2 mixtures were prepared. Mixture A: 18 μl GenePorter 2 plus 81 μl Optimem; Mixture B: 2 μg plasmid DNA plus 100 μl Diluent B (Gene Therapy Systems, San Diego, Calif.). Then master mixture was prepared by adding 33 μl Mixture A to 66 μl mixture B, and incubated for 5-15 minutes. Finally, 14 μl of the master mixture was added to the cells in each well.

Five hours later, the medium with transfection mixture in each well was replaced with Pen/Strp-free DMEM plus 2% FBS. Gamma secretase inhibitors were also added to the cells for inhibition studies.

### Example 7

### Transfection with Nucleofector II

About 5 to 10 millions (OR 1 to 10 millions) of cells were harvested from T-150 plates, and collected by centrifugation at 200×g for 7 minutes. Then cell pellet was rinsed with 10 ml of warm RPMI medium, and centrifuged again at 200×g for 5 minutes. Next, cell pellet was resuspended in 100 μl Solution R. To this cell suspension, 1-2 μg DNA was added, and the cell-DNA mixture was electroporated right away with a preset program T-20 on the Amaxa electroporation device (Amaxa Inc., Gathersberg, Md., USA). Once electroporation was done, 1 ml of room temperature RPMI was added to the electroporated cells. 2-5 minutes after addition of RPMI, the mixture was transferred into 5-10 ml of DMEM with 10% FBS, and plated into 96-well plates. One to three hours later, gamma secretase inhibitors were added to the cells for inhibition studies.

Table I summarizes the results obtained using a number of known γ-secretase inhibitor compounds. For example, several tested compounds are sulfonamide compounds, while several are non-sulfonamide compounds. The ratio of the EC₅₀ value obtained for Presenilin-2 transfected cells and Presenilin-1 transfected cells (indicated in the last column of Table I) indicates the degree to which the test compound is capable of preferentially inhibiting Presenilin-1. For example, Table I indicates that the sulfonamide compounds tested are 1.5- to 61-fold more potent at inhibiting Presenilin-1-comprised γ-secretase relative to Presenilin-2-comprised γ-secretase, and that the non-sulfonamide compounds tested were only 1.5 to 2-fold more potent. In Table 2, the values shown in columns A, B and C are EC₅₀ values (nM). Where inhibition was very low, EC₅₀ values were not generated by the program; thus EC₅₀ values are not provided. Rather, percent of inhibition was estimated based on the inhibition curve generated by the program. Percentages indicate percentage inhibition at a compound concentration of 10 uM.

### Example 8

### Identification of the Structural Basis for PS1 Selectivity of Small Inhibitor

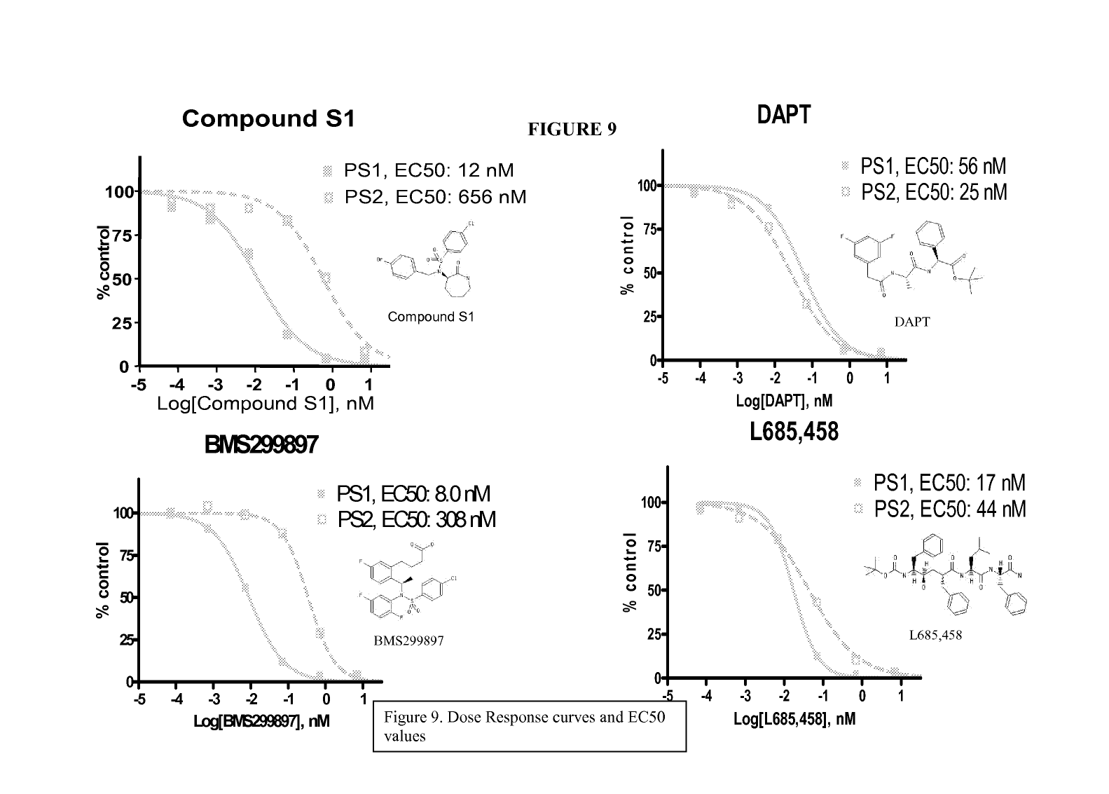

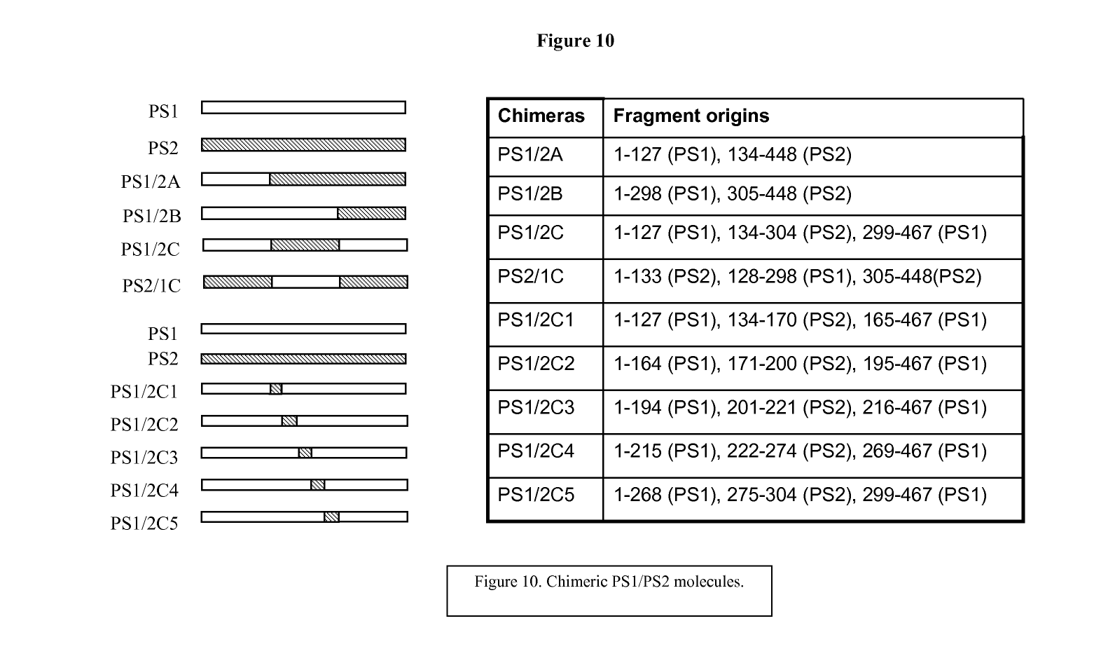

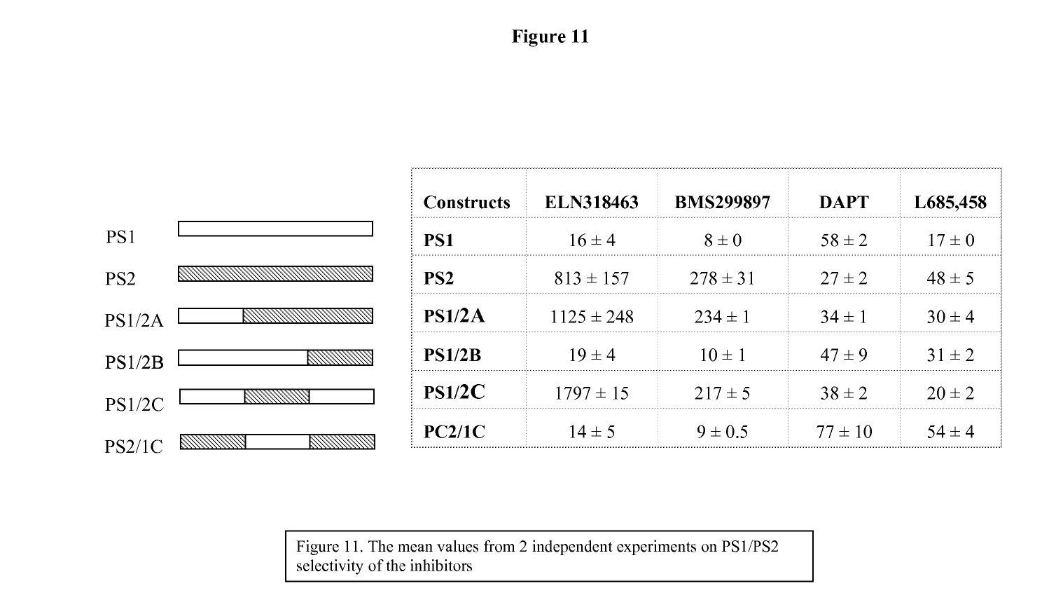

As discussed above, certain small molecule inhibitors, in particular, the sulfonamides, show preferential inhibition of PS1-γ-secretase, while non-sulfonamide inhibitors only have modest selectivity for PS1-vs. PS2-γ-secretase. The dose response curves and EC50 values from a representative experiment are shown in FIG. 9. The mean values from 2 independent experiments on PS1/PS2 selectivity of the inhibitors are shown in FIG. 11. COMPOUND S-1 is ˜51-fold more selective for PS1, and BMS299897 is ˜35-fold more selective for PS1, while L-685,458 is only ˜3-fold more selective for PS1, and DAPT is actually 2-fold more selective for PS2. Additional sulfonamide inhibitors of the type represented by Compound S-1 also displayed preferential PS1selectivity (data not shown). The observation of the differential inhibition of PS1 versus PS2, mainly by sulfonamide series of inhibitors, prompted us to examine the structural basis for this differential inhibition. We employed chimeric PS1/PS2 molecules (illustrated in FIG. 10) to map the domain(s) in PS1 responsible for differences in inhibitor potencies. Evaluation of an initial set of chimeric presenilin molecules revealed that the middle third of PS1 (residues 128-298) is both necessary and sufficient for its high potency inhibition by Compound S-1 and BMS299897 (FIG. 11). For both Compound S-1 and BMS299897, the EC50 values of PS1/2B are similar to that of PS1, while EC50 values of PS1/2A and PS1/2C are similar to those of PS2. More telling, inhibitor potencies against PS2/1C behaved just like PS1, in terms of its inhibition by Compound S-1and BMS299897, despite the fact that majority of this construct is comprised of PS2 sequence. As before (FIG. 9) non-sulfonamide inhibitors, such as DAPT and L-685,458, did not display >3-fold selectivity for PS1 nor PS2, and the chimeras did not reveal any consistent basis for this low level of selectivity. Further detailed analysis (using techniques that employ chimeric constructs and point mutations) identified amino acid residues L172, T281 and L282 of PS1 as being necessary and sufficient for selective inhibition of PS1 by Compound S-1. These residues also contributed in part to the PS1selective inhibition by BMS299897.

It should be understood that the foregoing disclosure emphasizes certain specific embodiments of the invention and that all modifications or alternatives equivalent thereto are within the spirit and scope of the invention as set forth in the appended claim

TABLE 2
PS1 and PS2 selectivity of gamma-secretase inhibitors

| | | | PS1 EC₅₀ (nM) | | |
|---|---|---|---|---|---|
| | | 293sw | | | |
| # | Batch | IC50(nM) | A | B | C |
| 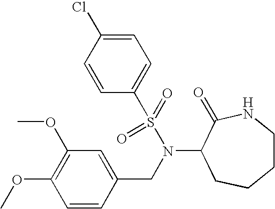 | 1 | | 228 | 425 | |
| 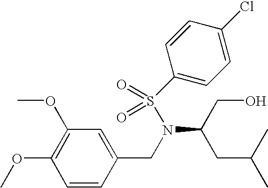 | 1 | 123 | 385 | 364 | |
| 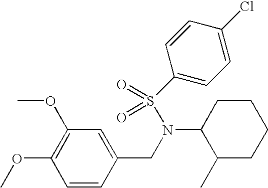 | 1 | 55 | 49 | 48 | |
| 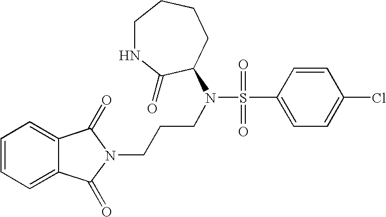 | 1 | 221 | 3715 | 6963 | |
| 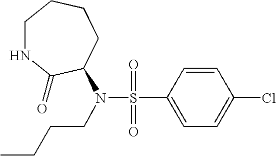 | 1 | 192 | 1407 | 928 | |
| 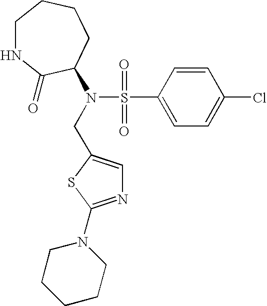 | 1 | 121 | 370 | 263 | |
| 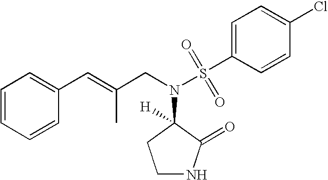 | 1 | 337 | 2171 | 1976 | |
| 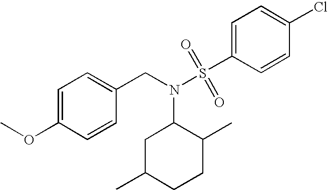 | 1 | 176 | 136 | 161 | |
| 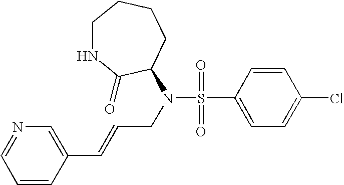 | 1 | 42 | 46 | 65 | |
| 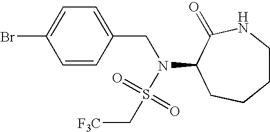 | 1 | 308 | 1283 | 686 | |
| 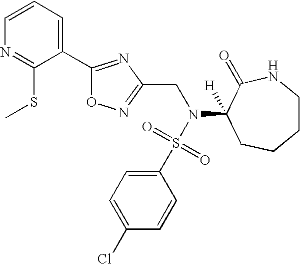 | 1 | 112 | 212 | 116 | |
| 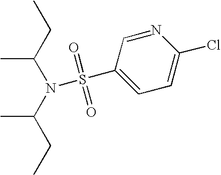 | 1 | 388 | 1392 | 787 | |
| 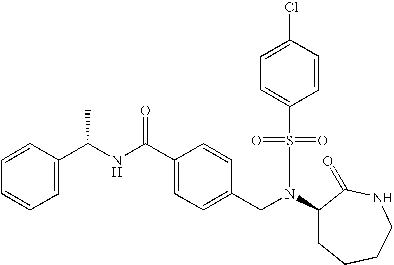 | 1 | | 143 | 139 | |
| 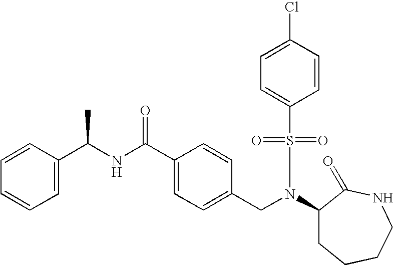 | 1 | 16 | 57 | 44 | |
| 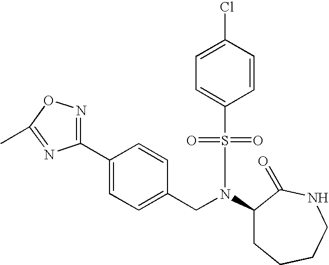 | 1 | 44 | 105 | 98 | |
| 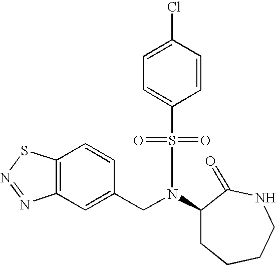 | 1 | 8 | 18 | 23 | |
| 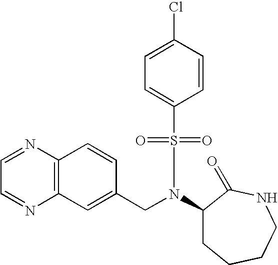 | 1 | 9 | 36 | 31 | |
| 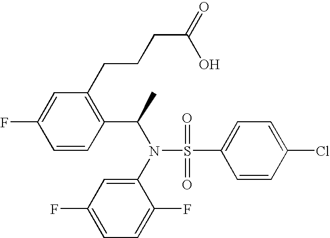 | 1 (BMS) | | 21 | 17 | |
| 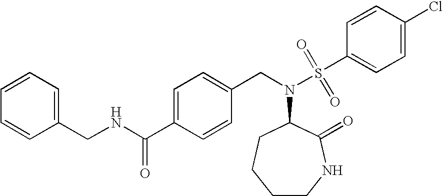 | 1 | 10 | 19 | 19 | |
| 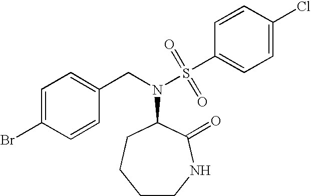 | 2 | 243 | 106 | 259 | 159 |
| 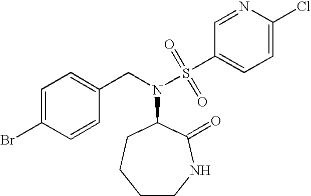 | 1 | 118 | 92 | 189 | 87 |
| 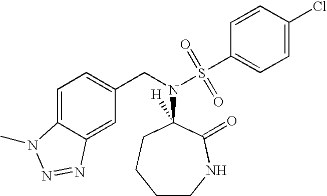 | 2 | 51 | 40 | 75 | 42 |
| 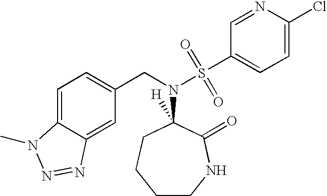 | 1 | 238 | 84 | 53 | 114 |
| 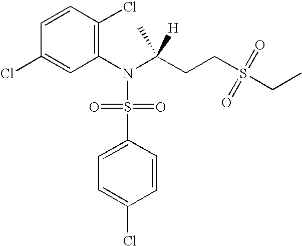 | 1 | 182 | 153 | 868 | 598 |
| 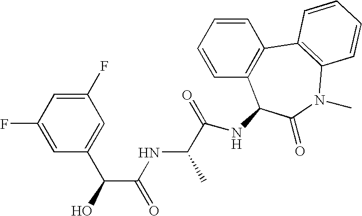 | | 0.064 | 0.6 | 0.39 | 0.74 |
| 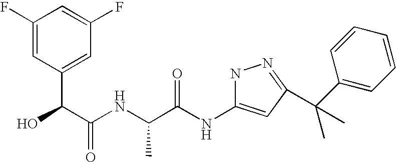 | 7 | 1 | 8.2 | 8.3 | 19 |
| 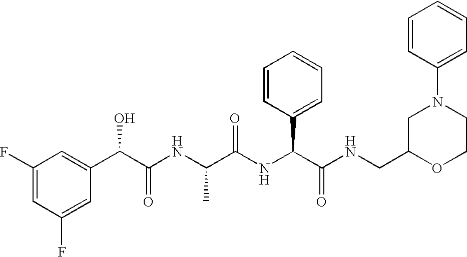 | 1 | 41 | 10 | 37 | 17.5 |
| 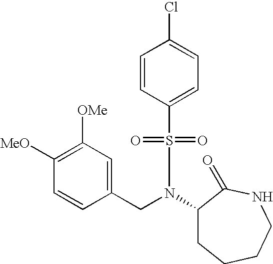 | 2 | 10000 | >10 μM | >10 μM | |
| 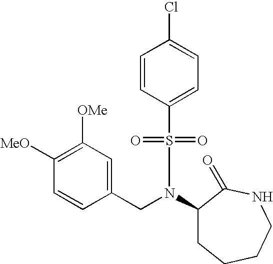 | 2 | 126 | 91 | 89 | 109 |
| 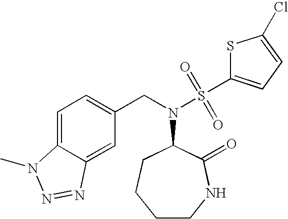 | 1 | 775 | 122 | 87 | 102 |
| 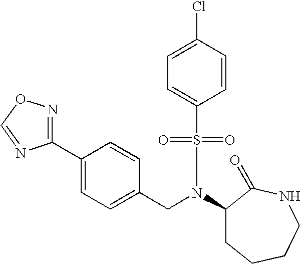 | 2 | 16 | 20 | 5.9 | 26 |
| 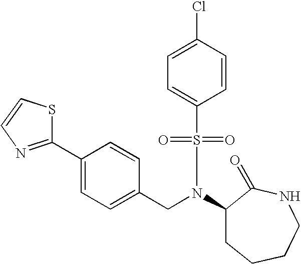 | 1 | 77 | 30 | 50 | 74 |
| 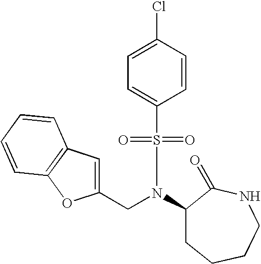 | 1 | | 30 | 63 | 50 |

| | PS2 EC₅₀* (nM) | | | |
|---|---|---|---|---|
| | | | | PS2/PS1 |
| # | A | B | C | EC₅₀ ratio |
| 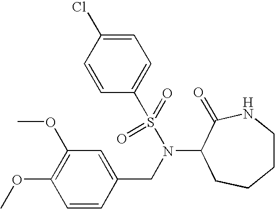 | *45% | *50% | | ˜30 |
| 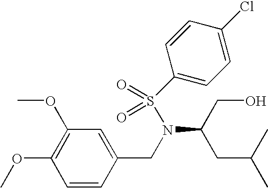 | *47% | *48% | | ˜26 |
| 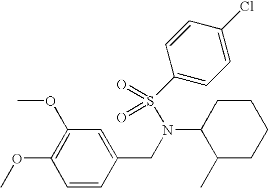 | 606 | 404 | | 10 |
| 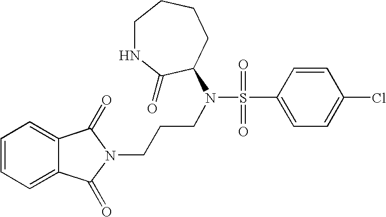 | *28% | *4% | | >10 |
| 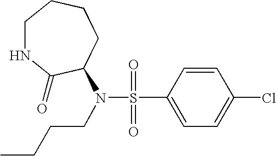 | *26% | *15% | | >10 |
| 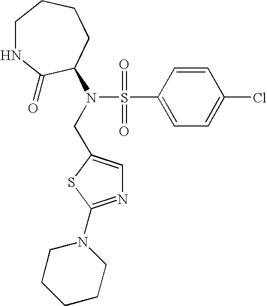 | *47% | *44% | | ˜30 |
| 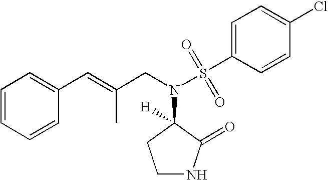 | *1% | *0% | | >10 |
| 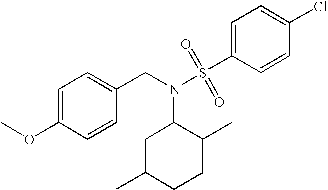 | 3000 | 3459 | | 22 |
| 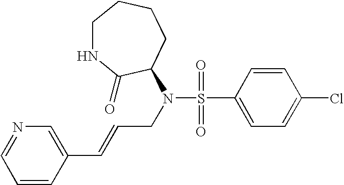 | 4004 | 2776 | | 61 |
| 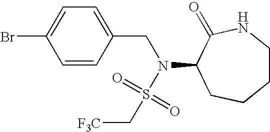 | *20% | *19% | | >10 |
| 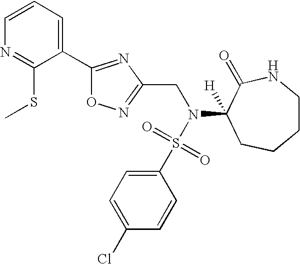 | 2100 | 2584 | | 11 |
| 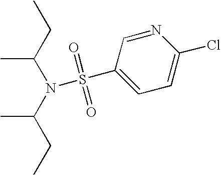 | 5862 | 3320 | | 4 |
| 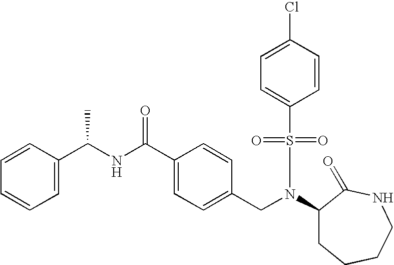 | 465 | 777 | | 4 |
| 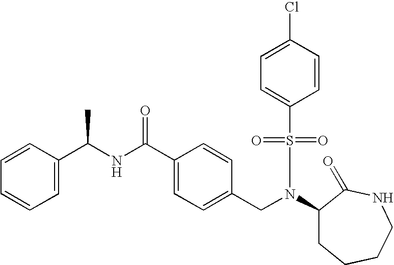 | 60 | 91 | | 1.5 |
| 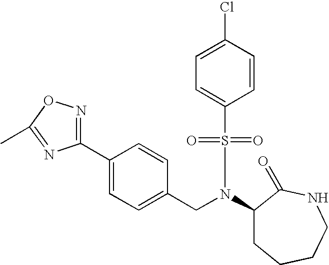 | 750 | 809 | | 8 |
| 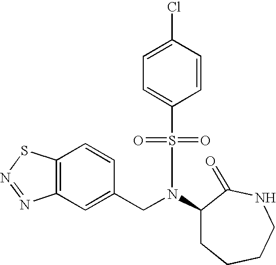 | 922 | 951 | | 47 |
| 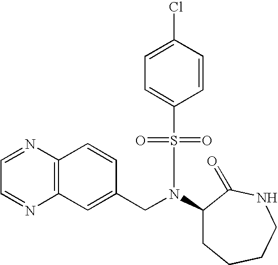 | 1411 | 1038 | | 37 |
| 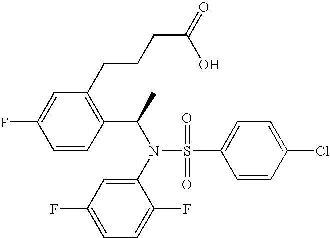 | 223 | 247 | | 12 |
| 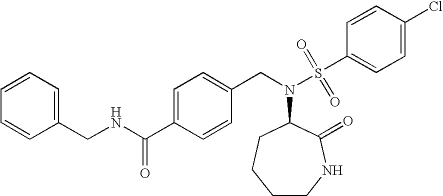 | 30 | 38 | | 1.8 |
| 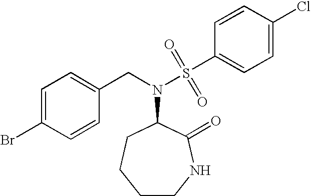 | 1919 | 3409 | | 15 |
| 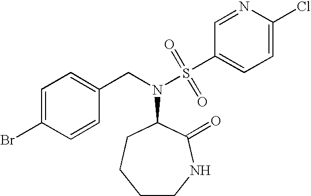 | 1566 | 2288 | | 31 |
| 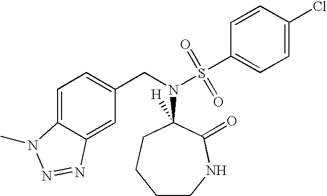 | 589 | 924 | | 15 |
| 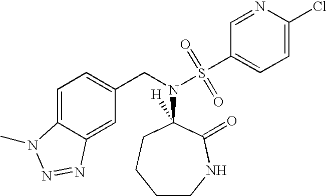 | 1981 | 1841 | | 23 |
| 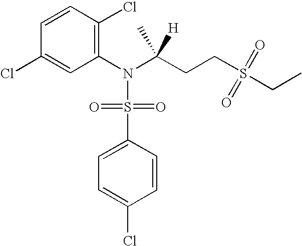 | 4998 | 5698 | | 10 |
| 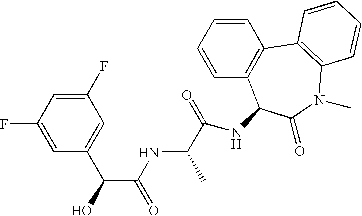 | 0.89 | 0.83 | | 1.5 |
| 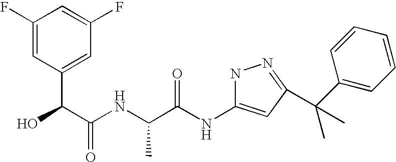 | 26 | 23 | | 2 |
| 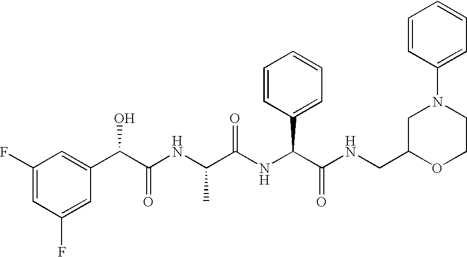 | 45 | 44 | | 2 |
| 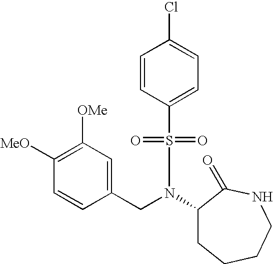 | >10 μM | >10 μM | | n/d |
| 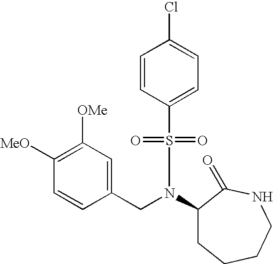 | 1289 | 1979 | 4545 | 27 |
| 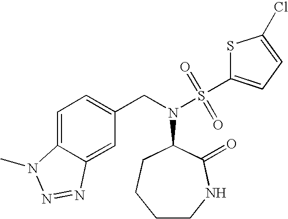 | 6411 | 5311 | 3000 | 47 |
| 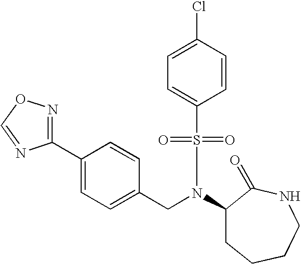 | 175 | 156 | 263 | 12 |
| 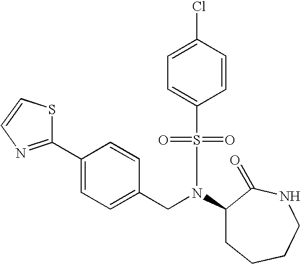 | 585 | 412 | 553 | 10 |
| 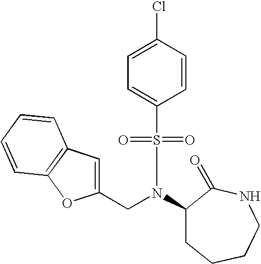 | 2418 | 1884 | 2376 | 46 |

What is claimed is:

1. A method for determining whether a compound preferentially inhibits Presenilin-1-comprised γ-secretase relative to Presenilin-2-comprised γ-secretase, comprising:
(a) separately incubating a first cell type that expresses Presenilin-1 but does not express Presenilin-2 and a second cell type that expresses Presenilin-2 but does not express Presenilin-1 with the compound;
(b) determining the amount of A40/42 in each cell line;
(c) calculating the EC₅₀ value for Aβ40/42 in each cell line; and
(d) determining that the compound preferentially inhibits Presenilin-1-comprised γ-secretase relative to Presenilin-2-comprised γ-secretase if the EC₅₀ value calculated for the first cell type is smaller than the EC₅₀ value calculated for the second cell type.

2. The method of claim 1, wherein the first cell type is a Presenilin-1/Presenilin-2 double knockout cell line transfected with a vector comprising Presenilin-1 cDNA and the second cell type is a Presenilin-1/Presenilin-2 double knockout cell line transfected with a vector comprising Presenilin-2.

3. A compound identified by the method of claim 1.

4. A pharmaceutical composition for treating Alzheimer's disease comprising a non-toxic therapeutically effective amount of the compound of claim 3 and a pharmaceutically acceptable carrier.

5. A method of treating Alzheimer's disease comprising administering to a patient in need thereof the pharmaceutical composition of claim 4.

6. A method for determining whether a sulfonamide compound preferentially inhibits Presenilin-1-comprised γ-secretase relative to Presenilin-2-comprised γ-secretase, comprising:
(a) separately incubating a first cell type that expresses Presenilin-1 but does not express Presenilin-2 and a second cell type that expresses Presenilin-2 but does not express Presenilin-1 with the compound;
(b) determining the amount of Aβ40/42 in each cell line;
(c) calculating the EC₅₀ value for Aβ40/42 in each cell line; and
(d) determining that the compound preferentially inhibits Presenilin-1-comprised γ-secretase relative to Presenilin-2-comprised γ-secretase if the EC₅₀ value calculated for the first cell type is smaller than the EC₅₀ value calculated for the second cell type.

7. The method of claim 6, wherein the first cell type is a Presenilin-1/Presenilin-2 double knockout cell line transfected with a vector comprising Presenilin-1 cDNA and the second cell type is a Presenilin-1/Presenilin-2 double knockout cell line transfected with a vector comprising Presenilin-2.

8. A compound identified by the method of claim 6.

9. A pharmaceutical composition for treating Alzheimer's disease comprising a non-toxic therapeutically effective amount of the compound of claim 8 and a pharmaceutically acceptable carrier.

10. A method of treating Alzheimer's disease comprising administering to a patient in need thereof the pharmaceutical composition of claim 9.

11. A method of selectively inhibiting PS1 relative to PS2 in a cell comprising administering the pharmaceutical composition of claim 9.

12. An isolated antibody that specifically binds to PS1, wherein said specific binding modulates the activity of presenilin 1-comprised gamma secretase (PS1).

13. The antibody of claim 12, wherein the antibody binds to the N-terminal portion of (PS1).

14. The antibody of claim 12, wherein the antibody binds to the N-terminal half of (PS1).

15. The antibody of claim 12, wherein the antibody does not bind to Presenilin-2.

16. The antibody of claim 12, wherein the antibody binds to the N-terminal sixth of PS1.

17. The antibody of any of claims 12, wherein said specific binding causes a reduction in the production of Aβ.

18. An isolated antibody having specific binding activity for Presenilin-1 (PS1) or a fragment thereof, wherein the antibody does not bind to Presenilin-2.

19. The specific binding agent of claim 18, wherein the isolated antibody has specific binding activity for SEQ ID NO: 8 or a fragment thereof.

20. The specific binding agent of claim 18, wherein the fragment of PS1 comprises at least 5 contiguous amino acids of PS1.

21. The specific binding agent of claim 20, wherein a portion of the at least 5 contiguous amino acids of PS1 are located in the N-terminal half of PS1.

22. The specific binding agent of claim 21, wherein the portion of the at least 5 contiguous amino acids of PS1 are located in the amino acid sequence of SEQ ID NO: 8.

23. An isolated polypeptide consisting of SEQ ID NO: 8.

24. A method for specifically inhibiting PS1, comprising contacting PS1 with a compound that binds to the N-terminal half of PS1 in an amount effective for specific inhibition.

25. The method of claim 24, wherein the compound binds to the N-terminal third of PS1.

26. The method of claim 24, wherein the compound binds to the N-terminal sixth of PS1.

27. The method of claim 24, wherein the contacting is performed in a cell.

28. The method of claim 27, wherein the cell is in vitro.

29. The method of claim 27, wherein the cell is a cell in culture.

30. The method of claim 27, wherein the compound does not inhibit activity of presenilin 2-comprised gamma secretase.

31. The method of claim 27, wherein the contacting causes a reduction in the production of A.

32. A method of treating or preventing Alzheimer's disease (AD) in a subject comprising administering to the subject an amount effective to treat or prevent AD of a specific-binding agent having specific binding activity for PS1, or pharmaceutically acceptable salts thereof.

33. A composition comprising a specific-binding agent having specific binding activity for PS1 in combination with a pharmaceutically acceptable salt, carrier, diluent, or adjuvant.

34. A method of treating or preventing Alzheimer's disease (AD) in a subject comprising administering to the subject an amount effective to treat or prevent AD of the composition of claim 33.

35. The method of claim 34, wherein the subject is a mammal.

36. The method of claim 35, wherein the mammal is a human.

37. An isolated polypeptide consisting of SEQ ID NO: 7.

38. A method of inhibiting the production of Aβ comprising contacting a cell that comprises PS1 and PS2 with a specific-binding agent having specific binding activity for PS1in an effective amount to inhibit PS1 gamma secretase activity and not inhibit PS2 gamma secretase activity.

39. The method of claim 38, wherein the contacting is in vitro.

40. The method of claim 38, wherein the contacting is in cell culture.

41. The method of claim 38, wherein the said contacting is in vivo.

42. A method of identifying a compound that inhibits PS1 activity, comprising: contacting a presenilin chimera constructed with an N terminal portion of PS1 with said compound, and measuring the relative activity of said chimera.

43. The method of claim 42, wherein the presenilin chimera comprises the amino acid sequence of SEQ ID NO: 8.

44. The method of claim 42, wherein the presenilin chimera comprises the amino acid sequence of SEQ ID NO: 7.

45. A method of identifying a compound that preferentially inhibits PS1 activity relative to PS2, comprising:
a) providing a first cell type that expresses PS1 but not PS2;
b) providing a second cell type that expresses PS2 but not PS1;
c) contacting the first cell type with a test compound;
d) contacting the second cell type with the same test compound;
e) determining an amount of Aβ peptide in the first and second cell type;
f) calculating an EC₅₀ for each cell type based on the amount Aβ peptide in the each cell type;
g) identifying the test compound as a compound that preferentially inhibits PS1activity if the EC₅₀ for the first cell type is smaller than the EC₅₀ for the second cell type.

46. The method of claim 45, wherein the Aβ peptide is Aβ38.

47. The method of claim 45, wherein the Aβ peptide is Aβ40.

48. The method of claim 45, wherein the Aβ peptide is Aβ42.

49. An isolated polypeptide consisting of SEQ ID NO: 9.

50. the specific binding agent of claim 18, wherein the isolated antibody has specific binding activity for SEQ ID NO: 9 or a fragment thereof.


SEQUENCE LISTING (9 sequences)

SEQ ID NO: 1 - DNA, 1404 bp, Homo sapiens
atggtggagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctag

SEQ ID NO: 2 - protein, 467 aa, Homo sapiens
MVELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 3 - DNA, 1347 bp, Homo sapiens
atggtgacattcatggcctctgacagcgaggaagaagtgtgtgatgagcggacgtccctaatgtcggccgagagccccacgccgcgctcctgccaggagggcaggcagggcccagaggatggagagaatactgcccagtggagaagccaggagaacgaggaggacggtgaggaggaccctgaccgctatgtctgtagtggggttcccgggcggccgccaggcctggaggaagagctgaccctcaaatacggagcgaagcatgtgatcatgctgtttgtgcctgtcactctgtgcatgatcgtggtggtagccaccatcaagtctgtgcgcttctacacagagaagaatggacagctcatctacccgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatcttg

SEQ ID NO: 4 - protein, 449 aa, Homo sapiens
MVTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYPPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYIL

SEQ ID NO: 5 - protein, 43 aa, Homo sapiens
DAEFRHDSGYEYHHQKLVFFAEDVGSNKGAIIGLMVGGVVIAT

SEQ ID NO: 6 - protein, 752 aa, Homo sapiens
MVLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVNLDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 7 - protein, 70 aa, Homo sapiens
MVELPAPLSYFQNAQMSEDNHLSNTVRSQNDNRERQEHNDRRSLGHPEPLSNGRPQGNSRQVVEQDEEED

SEQ ID NO: 8 - protein, 128 aa, Homo sapiens
MVELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQR

SEQ ID NO: 9 - protein, 171 aa, Homo sapiens
RALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNM